(19) United States
(12) Patent Application Publication
(10) Pub. No.: US 2026/0335389 A1
(43) Pub. Date: Oct. 29, 2026

(54) PROCESS FOR SYNTHESIZING A PHYTOTHERAPEUTIC COMPOSITION COMPRISING BIOACTIVE RESIN-DERIVED CONSTITUENTS FROM BOSWELLIA SERRATA AND COMMIPHORA

(71) Applicant: Dudekula Baba BASHA, Al’Majmaah (SA)

(72) Inventor: Dudekula Baba BASHA, Al’Majmaah (SA)

(21) Appl. No.: 19/675,291

(22) Filed: May 12, 2026

(51) Int. Cl. A61K 36/324; A61K 9/10; A61K 31/56; A61K 31/57; A61K 36/328; A61K 47/14; A61K 47/24

(52) U.S. Cl. CPC A61K 36/324; A61K 9/10; A61K 31/56; A61K 31/57; A61K 36/328; A61K 47/14; A61K 47/24; A61K 2236/11; A61K 2236/15; A61K 2236/333; A61K 2236/35; A61K 2236/39; A61K 2236/51; A61K 2236/53; A61K 2236/55

(57) ABSTRACT

The present invention relates to a process and an integrated system for synthesizing a phytotherapeutic composition comprising bioactive resin-derived constituents obtained from Boswellia serrata and Commiphora mukul. The invention discloses a multi-stage, parameter-controlled process involving resin pre-conditioning, solvent extraction under regulated temperature and agitation conditions, staged filtration, reduced-pressure concentration, selective fractionation, chromatographic purification, controlled blending, and high-shear as well as high-pressure homogenization. The process further includes incorporation of pharmaceutically acceptable carrier media and stabilization steps to produce a composition having enhanced bioavailability, reduced particle size, and improved physicochemical stability.

### TECHNICAL FIELD OF THE INVENTION

The present invention relates to the field of phytopharmaceutical processing and formulation engineering, and more particularly to a technically controlled process and an associated mechanical system for extraction, purification, stabilization, and formulation of bioactive resin-derived compounds obtained from Boswellia serrata and Commiphora mukul. The invention specifically pertains to a structurally integrated extraction and processing device configured to enable controlled solvent interaction, thermal regulation, fractionation, chromatographic separation, and homogenization for producing a pharmaceutically stable phytotherapeutic composition exhibiting enhanced bioavailability and compositional uniformity.

### BACKGROUND OF THE INVENTION

Resin-derived phytoconstituents such as boswellic acids and guggulsterones are known for their anti-inflammatory, lipid-modulating, and therapeutic properties. However, conventional extraction and formulation methods suffer from inconsistent yield, degradation of thermolabile constituents, residual solvent contamination, and poor bioavailability due to inadequate particle size control and dispersion instability. Furthermore, traditional batch processing lacks precise control over parameters such as solvent diffusion kinetics, temperature gradients, and shear-induced homogenization, thereby resulting in variability in the final composition.

Existing systems also fail to provide an integrated mechanical architecture capable of performing sequential operations including extraction, filtration, fractionation, chromatographic purification, and nano-scale homogenization within a controlled environment. The absence of such integrated systems leads to process inefficiencies, contamination risks, and difficulty in scaling.

The present invention addresses these limitations by providing a highly controlled process along with a structurally defined device comprising interconnected vessels, regulated fluid pathways, and precision-controlled processing units configured to ensure reproducibility, enhanced extraction efficiency, and improved physicochemical stability of the final phytotherapeutic composition.

The development of phytotherapeutic compositions derived from natural resins such as those obtained from Boswellia serrata and Commiphora mukul has attracted considerable scientific and industrial interest due to their well-documented pharmacological properties, including anti-inflammatory, anti-arthritic, lipid-lowering, and immunomodulatory effects. The principal bioactive constituents present in these resins, particularly boswellic acids and guggulsterones, have been widely investigated for therapeutic applications in chronic inflammatory disorders, metabolic syndromes, and cardiovascular conditions. Despite the promising therapeutic potential, the translation of these natural compounds into stable, standardized, and bioavailable pharmaceutical formulations remains a significant technical challenge, primarily due to limitations associated with conventional extraction, purification, and formulation methodologies.

Traditional extraction techniques for resin-derived phytoconstituents largely rely on maceration, Soxhlet extraction, or simple reflux-based solvent extraction methods. While these techniques are relatively straightforward and cost-effective, they suffer from several inherent drawbacks. One major limitation is the lack of precise control over extraction parameters such as temperature, solvent polarity, agitation, and extraction duration, which directly influences the yield and composition of extracted bioactive compounds. For instance, prolonged exposure to elevated temperatures during Soxhlet extraction can result in thermal degradation of thermolabile constituents such as acetylated boswellic acids, thereby reducing their pharmacological efficacy. Similarly, insufficient control over solvent composition in hydroalcoholic extraction processes often leads to co-extraction of undesirable resinous impurities, pigments, and waxes, which complicate downstream purification steps and negatively affect the purity of the final product.

Another significant limitation of existing extraction processes is the inefficient mass transfer between the resin matrix and the solvent system. Resin materials are inherently viscous and heterogeneous, which restricts solvent penetration and limits the diffusion of target compounds into the extraction medium. Conventional methods typically do not incorporate pre-conditioning steps such as controlled humidity exposure or staged solvent addition, which could otherwise enhance resin swelling and improve extraction efficiency. As a result, these processes often yield extracts with inconsistent concentrations of active constituents, leading to batch-to-batch variability that is unacceptable in pharmaceutical-grade products.

Following extraction, purification of boswellic acids and guggulsterones presents additional technical challenges. Current purification approaches commonly involve liquid-liquid extraction, open-column chromatography, or recrystallization techniques. While silica-based column chromatography is effective in isolating specific fractions, it is highly labor-intensive, time-consuming, and difficult to scale for industrial production. Moreover, conventional chromatographic systems lack automated control over solvent gradients, flow rates, and fraction collection, resulting in poor reproducibility and significant material losses. Liquid-liquid partitioning methods, although simpler, often suffer from incomplete separation of structurally similar compounds, leading to impurities in the final extract. Additionally, repeated solvent usage in these processes increases the risk of residual solvent contamination, which poses safety and regulatory concerns.

The concentration and solvent removal stages in existing processes also exhibit notable inefficiencies. Rotary evaporation systems are widely used; however, they often operate without precise control of pressure and temperature cycling, which can result in localized overheating and degradation of sensitive phytoconstituents. Furthermore, inadequate removal of residual solvents can compromise the stability and safety of the final formulation. In many conventional setups, the absence of inert atmospheric conditions during concentration exposes the extracts to oxygen, leading to oxidative degradation and loss of bioactivity over time.

A further critical limitation lies in the formulation and stabilization of the extracted bioactive compounds. Boswellic acids and guggulsterones are inherently hydrophobic and exhibit poor aqueous solubility, which significantly limits their bioavailability when administered in conventional dosage forms such as tablets or capsules. Existing formulation strategies often rely on simple blending with excipients or incorporation into oil-based carriers, which fail to achieve uniform dispersion or nanoscale particle size reduction. As a consequence, the absorption of active constituents in the gastrointestinal tract remains suboptimal, resulting in reduced therapeutic efficacy.

Attempts have been made to improve bioavailability through advanced delivery systems such as liposomes, nanoemulsions, and solid lipid nanoparticles. However, these approaches typically require complex multi-step processing, specialized equipment, and stringent control over formulation parameters. In many cases, the lack of integration between extraction and formulation stages leads to incompatibility issues, where the physicochemical properties of the extracted compounds are not optimized for subsequent encapsulation or dispersion. Additionally, high-shear homogenization and high-pressure processing techniques used in such systems often generate excessive heat, which can degrade sensitive phytochemicals if not properly controlled.

From a systems engineering perspective, one of the most significant drawbacks of existing technologies is the absence of an integrated processing platform capable of performing all stages of production in a continuous and controlled manner. Conventional manufacturing setups typically involve discrete units for extraction, filtration, concentration, purification, and formulation, with manual transfer of intermediate materials between stages. This not only increases the risk of contamination but also leads to process inefficiencies, higher operational costs, and difficulty in maintaining aseptic conditions. Moreover, the lack of real-time monitoring and feedback control mechanisms prevents dynamic adjustment of processing parameters, resulting in suboptimal product quality.

Another challenge associated with existing solutions is scalability. Processes that are effective at the laboratory scale often fail to deliver consistent results when scaled up for industrial production. This is particularly evident in chromatographic purification and nano-formulation techniques, where maintaining uniform flow distribution, pressure conditions, and particle size becomes increasingly difficult at larger volumes. The absence of robust mechanical systems designed specifically for large-scale phytochemical processing further exacerbates this issue.

Environmental and regulatory considerations also highlight the shortcomings of conventional methods. Extensive use of organic solvents such as hexane, chloroform, and petroleum ether raises concerns regarding solvent recovery, emissions, and operator safety. Many existing processes lack efficient solvent recycling mechanisms, leading to increased waste generation and environmental impact. Additionally, stringent regulatory requirements for residual solvent levels and product purity necessitate advanced processing controls that are not adequately addressed by traditional methods.

While numerous techniques have been developed for the extraction and formulation of resin-derived phytotherapeutic compounds, they are characterized by significant limitations including poor control over extraction parameters, inefficient mass transfer, inadequate purification, thermal and oxidative degradation, low bioavailability, lack of process integration, scalability challenges, and environmental concerns. These drawbacks underscore the need for a technically advanced process and a structurally integrated system capable of overcoming these limitations through precise control of mechanical, thermal, and chemical parameters across all stages of production. The present invention addresses these challenges by providing a comprehensive solution that integrates extraction, purification, and formulation within a unified system, thereby enabling the production of high-quality, stable, and bioavailable phytotherapeutic compositions suitable for pharmaceutical applications.

### SUMMARY OF THE INVENTION

The invention provides a multi-stage process for synthesizing a phytotherapeutic composition enriched in boswellic acids and guggulsterones, wherein each stage is controlled with respect to solvent composition, temperature, agitation, pressure, and purification parameters. The process is implemented using a specially designed mechanical system comprising extraction vessels, filtration assemblies, reduced-pressure concentration units, fractionation chambers, chromatographic columns, blending vessels, and high-shear homogenization units.

The invention further provides a structurally integrated device configured to facilitate sequential processing of resin materials through controlled mechanical and thermodynamic environments, thereby ensuring enhanced extraction efficiency, selective enrichment of active constituents, and formation of a stable nano-dispersed composition.

The primary object of the present invention is to provide a technically advanced and highly controlled process for synthesizing a phytotherapeutic composition comprising bioactive resin-derived constituents from Boswellia serrata and Commiphora mukul, wherein the process enables efficient extraction, selective enrichment, and stabilization of boswellic acids and guggulsterones while minimizing degradation, impurity carryover, and variability in composition. The invention seeks to achieve reproducible extraction efficiency by regulating critical parameters including solvent composition, temperature, agitation, pressure, and extraction duration, thereby ensuring consistent yield and compositional uniformity suitable for pharmaceutical applications.

Another object of the present invention is to provide a process that enhances mass transfer and solvent-resin interaction through controlled pre-conditioning, staged solvent addition, and optimized agitation regimes, thereby improving diffusion kinetics and maximizing recovery of desired bioactive constituents. The invention further aims to provide a purification methodology that enables selective isolation of targeted compounds such as 3-acetyl-11-keto-β-boswellic acid and E- and Z-guggulsterones using controlled fractionation and chromatographic separation techniques, thereby improving purity levels and reducing the presence of non-therapeutic or interfering components.

A further object of the invention is to provide a process for producing a phytotherapeutic composition with enhanced physicochemical stability and bioavailability by incorporating the purified bioactive fractions into a carrier medium under controlled homogenization conditions, including high-shear and high-pressure processing, such that the resulting composition exhibits reduced particle size, uniform dispersion, and improved absorption characteristics. The invention also aims to minimize residual solvent content and oxidative degradation through controlled concentration, inert atmosphere handling, and stabilization steps including pH adjustment and antioxidant incorporation.

Another object of the present invention is to provide a structurally integrated system configured as a multi-unit mechanical apparatus capable of performing extraction, filtration, concentration, fractionation, chromatographic purification, blending, and homogenization in a sequential and controlled manner, thereby eliminating the need for manual transfer between stages and reducing contamination risks. The invention aims to ensure precise regulation of process variables across interconnected units through a programmable control mechanism, thereby improving process efficiency, reproducibility, and scalability.

A further object of the invention is to provide a device architecture that enables continuous or semi-continuous operation with integrated fluid pathways, temperature regulation systems, pressure control mechanisms, and agitation assemblies, thereby facilitating industrial-scale production of phytotherapeutic compositions with consistent quality. The invention also seeks to incorporate features for solvent recovery and recycling, thereby reducing environmental impact and improving process sustainability.

Another object of the invention is to provide a system and process that are adaptable to variations in raw material characteristics, including differences in resin composition, moisture content, and particle size, by enabling dynamic adjustment of processing parameters based on real-time monitoring. The invention further aims to provide a robust and scalable solution suitable for pharmaceutical and nutraceutical manufacturing environments requiring stringent quality control and regulatory compliance.

A further object of the present invention is to provide a phytotherapeutic composition characterized by a defined ratio of boswellic acids to guggulsterones, controlled particle size distribution, low residual solvent content, and enhanced stability, thereby ensuring improved therapeutic efficacy and shelf life. The invention also aims to provide flexibility in downstream processing, including drying, encapsulation, and formulation into various dosage forms, without compromising the integrity of the bioactive constituents.

Another object of the invention is to overcome the limitations of conventional extraction and formulation techniques by integrating mechanical, chemical, and thermodynamic control within a unified system, thereby enabling efficient, reliable, and economically viable production of high-quality phytotherapeutic products.

### BRIEF DESCRIPTION OF FIGURES

These and other features, aspects, and advantages of the present invention will become better understood when the following detailed description is read concerning the accompanying drawings in which like characters represent like parts throughout the drawings, wherein:

FIG. 1 displays a flow chart diagram of a process for synthesizing a phytotherapeutic composition comprising bioactive resin-derived constituents from Boswellia and Commiphora;

FIG. 2 displays block diagram of a system for synthesizing a phytotherapeutic composition comprising bioactive resin-derived constituents from Boswellia serrata and Commiphora;

FIG. 3 illustrates comparative inhibition of pro-inflammatory cytokines TNF-α and IL-6 in LPS-stimulated RAW 264.7 macrophage cells treated with different concentrations of the synthesized phytotherapeutic composition;

FIG. 4 illustrates comparative reduction in paw edema in Freund's complete adjuvant-induced arthritic Wistar rats following administration of the synthesized phytotherapeutic composition and a standard anti-inflammatory treatment; and

FIG. 5 illustrates accelerated stability analysis of the synthesized phytotherapeutic composition showing comparative values of boswellic acid concentration, guggulsterone concentration, particle size, and pH at initial and three-month storage conditions.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION OF THE INVENTION:

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

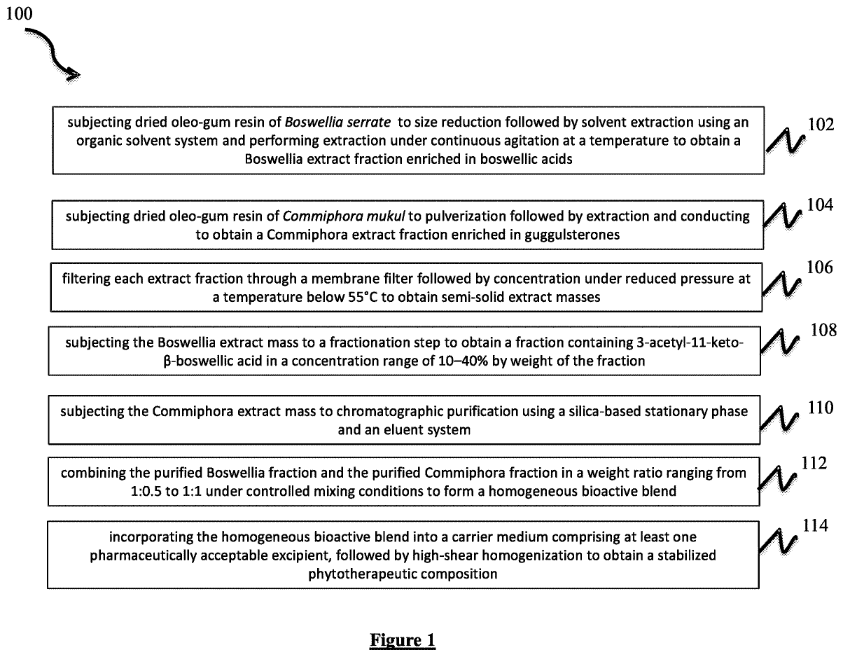

Referring to FIG. 1, a flow chart of a process for synthesizing a phytotherapeutic composition comprising bioactive resin-derived constituents from Boswellia and Commiphora, is illustrated. The process 100 comprising:

At step 102, the process 100 includes subjecting dried oleo-gum resin of Boswellia serrate to size reduction followed by solvent extraction using an organic solvent system comprising ethanol in a concentration range of 70-95% v/v, maintaining a solid-to-solvent ratio between 1:5 and 1:15 (w/v), and performing extraction under continuous agitation at a temperature maintained between 45 °C and 65 °C for a duration of 4 to 10 hours to obtain a Boswellia extract fraction enriched in boswellic acids;

At step 104, the process 100 includes subjecting dried oleo-gum resin of Commiphora mukul to pulverization (size reduction) followed by extraction using a hydroalcoholic solvent comprising ethanol and water in a volumetric ratio between 60:40 and 90:10, maintaining a solid-to-solvent ratio between 1:4 and 1:12 (w/v), and conducting extraction at a temperature between 40 °C and 60 °C for a duration of 3 to 8 hours to obtain a Commiphora extract fraction enriched in guggulsterones;

At step 106, the process 100 includes filtering each extract fraction through a membrane filter having a pore size between 0.2 μm and 5 μm followed by concentration under reduced pressure at a temperature below 55 °C to obtain semi-solid extract masses;

At step 108, the process 100 includes subjecting the Boswellia extract mass to a fractionation step using a non-polar solvent selected from hexane or petroleum ether to remove volatile terpenoids, followed by re-extraction with ethyl acetate to obtain a fraction containing 3-acetyl-11-keto-β-boswellic acid in a concentration range of 10-40% by weight of the fraction;

At step 110, the process 100 includes subjecting the Commiphora extract mass to chromatographic purification using a silica-based stationary phase and an eluent system comprising hexane and ethyl acetate in a ratio between 7:3 and 9:1 to isolate E-and Z-guggulsterones in a combined concentration range of 2-15% by weight of the fraction;

At step 112, the process 100 includes combining the purified Boswellia fraction and the purified Commiphora fraction in a weight ratio ranging from 1:0.5 to 1:2 under controlled mixing conditions at a temperature between 25 °C and 40 °C to form a homogeneous bioactive blend;

At step 114, the process 100 includes incorporating the homogeneous bioactive blend into a carrier medium comprising at least one pharmaceutically acceptable excipient selected from phospholipids, medium-chain triglycerides, or polysorbates in a concentration range of 5-40% w/w, followed by high-shear homogenization at a shear rate between 5,000 s⁻¹ and 20,000 s⁻¹ for a duration of 10 to 30 minutes to obtain a stabilized phytotherapeutic composition.

In an embodiment, the extraction of the Boswellia serrata resin(102) further comprises pre-conditioning the pulverized/size-reduced resin by exposing it to a controlled humidity environment maintained at 55-70% relative humidity and a temperature between 28 °C and 35 °C for a duration of 6 to 18 hours prior to solvent contact, followed by gradual addition of the ethanol solvent in a staged manner comprising an initial wetting phase using 20-30% of the total solvent volume introduced over 10-20 minutes under low-speed agitation between 50 and 150 rpm, and a subsequent extraction phase in which the remaining solvent volume is introduced continuously while increasing agitation speed to 300-600 rpm to facilitate controlled diffusion of boswellic acids into the solvent medium.

In an embodiment, the extraction of resin derived from Boswellia serrata is preceded by a controlled pre-conditioning stage designed to modify the physicochemical characteristics of the pulverized resin in a manner that enhances solvent accessibility and diffusion behavior during subsequent extraction. The pulverized resin, typically reduced to a particle size distribution within the range of 100 μm to 500 μm using a mechanical milling unit, is placed within a humidity-regulated conditioning chamber equipped with a closed-loop environmental control system capable of maintaining relative humidity between 55% and 70% and temperature between 28 °C and 35 °C The chamber includes a humidified air circulation mechanism, such as a vapor injection unit combined with a feedback-controlled hygrometer, ensuring uniform moisture exposure across the resin bed. Over a conditioning duration of 6 to 18 hours, the resin matrix absorbs controlled moisture, leading to partial plasticization and swelling of the gum-resin structure, thereby reducing internal mass transfer resistance and increasing porosity without causing hydrolytic degradation or microbial growth.

Following the conditioning phase, the resin is transferred to an extraction vessel configured with an impeller-driven agitation assembly and a temperature-regulated jacket. The solvent addition is performed in a staged manner governed by flow-controlled dosing units. In an initial wetting phase, approximately 20% to 30% of the total ethanol solvent volume, typically within a concentration range of 70% to 95% v/v, is introduced gradually over a period of 10 to 20 minutes while maintaining agitation at a low speed between 50 and 150 rpm. This controlled introduction ensures uniform wetting of the resin particles and prevents formation of agglomerates or localized solvent saturation zones, which could otherwise hinder diffusion pathways. The reduced agitation during this phase minimizes shear-induced compaction of the resin mass and allows capillary penetration of the solvent into the pre-conditioned porous structure.

Upon completion of the wetting phase, the extraction transitions into a dynamic diffusion phase in which the remaining solvent volume is continuously introduced into the vessel through a metered feed system while the agitation speed is progressively increased to a range between 300 and 600 rpm. The increase in agitation enhances convective mass transfer and ensures homogeneous mixing of the solvent-resin system, while the pre-conditioned state of the resin facilitates efficient release of boswellic acids into the solvent medium. The extraction temperature is maintained within a controlled range, typically between 45 °C and 65 °C, using the jacketed heating system, ensuring optimal solubility of triterpenoid compounds without inducing thermal degradation.

For example, in a representative operational setup, 2 kg of pre-conditioned Boswellia resin is subjected to staged extraction using 12 L of 90% ethanol, wherein 3 L is introduced during the wetting phase at 100 rpm over 15 minutes, followed by gradual addition of the remaining 9 L over 4 hours while increasing agitation to 450 rpm and maintaining temperature at 55 °C Analytical quantification using high-performance liquid chromatography demonstrates a higher concentration of 3-acetyl-11-keto-β-boswellic acid in the resulting extract compared to a non-conditioned, single-stage extraction process, indicating improved extraction efficiency and selectivity. The controlled sequence of moisture conditioning, staged solvent addition, and variable agitation ensures uniform extraction kinetics, minimizes solvent wastage, and produces an extract with enhanced compositional consistency suitable for subsequent purification and formulation stages.

In an embodiment, the extraction of the Commiphora mukul resin (104) includes a sequential dual-phase extraction protocol comprising an initial extraction stage using ethanol-water mixture at a ratio of 70:30 conducted at 40-45 °C for 2 to 4 hours to solubilize polar constituents, followed by separation of the solvent phase and re-extraction of the residual solid mass using ethanol concentration of 85-95% v/v at 50-60 °C for an additional 2 to 5 hours, wherein the combined extract fractions are subjected to controlled cooling at a rate of 1-3 °C per minute to a final temperature of 10-15 °C to induce precipitation of high molecular weight resinous impurities prior to filtration.

In an embodiment, the extraction of resin derived from Commiphora mukul is performed through a sequential dual-phase extraction protocol specifically configured to differentiate and selectively recover polar and non-polar bioactive constituents while minimizing co-extraction of undesired resinous materials. The process begins with pulverization of the dried oleo-gum resin to a controlled particle size, typically within the range of 100 μm to 600 μm, to enhance surface exposure and facilitate solvent interaction. The pulverized material is introduced into a temperature-controlled extraction vessel equipped with an agitation assembly and solvent dosing system. In the first phase, an ethanol-water mixture at a volumetric ratio of approximately 70:30 is introduced at a solid-to-solvent ratio typically between 1:6 and 1:10 (w/v), and the mixture is maintained at a temperature between 40 °C and 45 °C under continuous agitation at 200 to 400 rpm for a duration of 2 to 4 hours. Under these conditions, polar constituents including certain sterols, glycosidic fractions, and water-soluble components are preferentially solubilized due to the increased polarity of the solvent system, while non-polar resin fractions remain largely unextracted within the solid matrix.

Upon completion of the initial extraction phase, the solvent phase is separated from the residual solid mass using a decantation or filtration mechanism, ensuring minimal disturbance of the remaining resin matrix. The residual solid mass is then subjected to a second extraction phase using a higher concentration ethanol solvent, typically in the range of 85% to 95% v/v, at an elevated temperature between 50 °C and 60 °C This second phase is conducted for an additional duration of 2 to 5 hours under controlled agitation, typically between 300 and 600 rpm, to facilitate the extraction of non-polar bioactive constituents such as E-and Z-guggulsterones. The use of higher ethanol concentration reduces solvent polarity, thereby enhancing solubilization of lipophilic compounds while limiting extraction of residual hydrophilic impurities.

The extracts obtained from both phases are subsequently combined in a controlled manner and subjected to a cooling protocol designed to induce selective precipitation of high molecular weight resinous impurities. The combined extract is transferred to a cooling vessel equipped with a temperature regulation system capable of achieving a controlled cooling rate of 1 °C to 3 °C per minute. The temperature is gradually reduced from the extraction temperature to a final range between 10 °C and 15 °C, during which supersaturation conditions promote aggregation and precipitation of waxes, gums, and other high molecular weight fractions that exhibit reduced solubility at lower temperatures. The precipitation process is allowed to stabilize for a holding period of 30 to 90 minutes to ensure complete phase separation.

For example, in a representative implementation, 1.5 kg of pulverized Commiphora resin is subjected to initial extraction using 10 L of 70:30 ethanol-water mixture at 42 °C for 3 hours, followed by separation and re-extraction with 8 L of 90% ethanol at 55 °C for 4 hours. The combined extract is then cooled from 50 °C to 12 °C at a rate of approximately 2 °C per minute, resulting in visible precipitation of resinous impurities, which are subsequently removed by filtration using a 0.45 μm membrane filter. Analytical evaluation of the clarified extract demonstrates a higher relative concentration of guggulsterones with reduced background impurities compared to single-phase extraction methods. This sequential extraction and controlled cooling approach enables improved selectivity, enhances purity of the target compounds, and facilitates downstream chromatographic separation by reducing column fouling and improving fraction resolution.

In an embodiment, the concentration under reduced pressure is performed using a rotary evaporation system operating at a pressure between 80 and 150 mbar and a rotation speed between 60 and 120 rpm, wherein the solvent removal is conducted in incremental cycles each lasting 10-20 minutes with intermittent cooling phases of 5-10 minutes to maintain the internal temperature of the extract mass below 50 °C, followed by nitrogen purging at a flow rate between 0.5 and 2 L/min for 10 to 30 minutes to remove residual solvent traces and minimize oxidative degradation of bioactive constituents.

In an embodiment, the concentration stage following extraction of resin-derived fractions from Boswellia serrata and Commiphora mukul is carried out using a rotary evaporation system configured to enable controlled solvent removal under reduced pressure while preserving the structural integrity of thermally sensitive bioactive constituents. The clarified extract obtained after filtration is transferred into a rotating evaporation flask constructed of chemically resistant glass and coupled to a vacuum system capable of maintaining pressure within the range of 80 to 150 mbar. The flask is partially immersed in a temperature-controlled heating bath, typically maintained between 40 °C and 50 °C, and rotated at a speed between 60 and 120 rpm to generate a thin film of the extract along the inner wall surface, thereby increasing the effective surface area for evaporation and promoting efficient solvent removal.

The solvent removal process is not performed continuously but rather in a sequence of incremental evaporation cycles, each lasting between 10 and 20 minutes, to prevent localized overheating and excessive concentration gradients within the extract mass. During each cycle, the reduced pressure lowers the boiling point of the solvent system, enabling rapid evaporation at temperatures significantly below atmospheric boiling points. Following each evaporation interval, the system is subjected to an intermittent cooling phase of 5 to 10 minutes, during which heating is temporarily reduced or halted and, where applicable, a cooling medium is circulated through the bath or jacket to dissipate accumulated heat. This cyclic approach ensures that the internal temperature of the extract mass remains consistently below 50 °C, thereby preventing degradation, isomerization, or loss of volatile functional groups associated with boswellic acids and guggulsterones.

The progress of solvent removal is monitored through measurement of condensate volume collected in the receiving flask, as well as by observing changes in viscosity and mass of the extract. In certain implementations, inline sensors such as infrared or vapor composition analyzers may be used to estimate residual solvent concentration in real time. Once the solvent content is reduced to a semi-solid consistency, typically corresponding to residual solvent levels below approximately 5% w/w, the evaporation process is terminated, and the concentrated extract is subjected to an inert gas purging step.

The nitrogen purging stage is performed by introducing a controlled flow of nitrogen gas into the evaporation chamber or a separate sealed vessel containing the concentrated extract, at a flow rate between 0.5 and 2 L/min for a duration of 10 to 30 minutes. The nitrogen gas displaces residual solvent vapors and dissolved oxygen from the extract matrix, thereby reducing the likelihood of oxidative transformations and further facilitating removal of trace solvent molecules. The purging operation may be conducted under mild agitation or static conditions, depending on system configuration, and is typically performed at ambient or slightly elevated temperatures not exceeding 40 °C.

For example, in a representative implementation, 5 L of filtered extract containing dissolved boswellic acids is subjected to rotary evaporation at 100 mbar and 80 rpm, with bath temperature maintained at 45 °C The process is carried out in three evaporation cycles of 15 minutes each, interspersed with 7-minute cooling intervals, resulting in progressive reduction of solvent volume to approximately 0.5 L of viscous concentrate. Subsequent nitrogen purging at 1 L/min for 20 minutes further reduces residual solvent content to below detectable limits while maintaining compositional integrity. The resulting semi-solid extract exhibits improved stability and is suitable for downstream fractionation and formulation processes, demonstrating that controlled cyclic evaporation combined with inert gas purging provides a reliable and reproducible approach for solvent removal without compromising the quality of the bioactive constituents.

In an embodiment, the fractionation of the Boswellia extract mass (108)includes dispersing the concentrated extract in hexane at a ratio of 1:3 to 1:8 (w/v) and subjecting the dispersion to ultrasonic agitation at a frequency between 20 kHz and 40 kHz and power density between 100 and 300 W/L for 15 to 45 minutes to facilitate selective dissolution of non-polar terpenoids, followed by phase separation through centrifugation at 3,000 to 8,000 rpm for 10 to 25 minutes, and subsequent recovery of the insoluble fraction which is then re-extracted using ethyl acetate under reflux conditions at 60-75 °C for 1 to 3 hours to enrich boswellic acid concentration.

In an embodiment, the fractionation of the concentrated extract derived from Boswellia serrata is carried out through a solvent-selective separation protocol designed to remove non-polar terpenoid impurities while enriching the fraction in bioactive boswellic acids. The semi-solid extract obtained after reduced-pressure concentration is first dispersed in a non-polar solvent such as hexane at a controlled ratio ranging from 1:3 to 1:8 (w/v), wherein the extract is gradually introduced into the solvent under mild stirring to ensure uniform dispersion and to avoid formation of localized agglomerates. The dispersion is then subjected to ultrasonic agitation using an ultrasonic processor equipped with a probe or bath system operating within a frequency range of 20 kHz to 40 kHz and a power density between 100 and 300 W/L. The ultrasonic energy induces cavitation within the solvent medium, generating localized micro-turbulence and high-energy shear zones that enhance solvent penetration into the extract matrix and promote selective dissolution of low molecular weight and non-polar terpenoid components into the hexane phase.

The ultrasonic treatment is maintained for a duration between 15 and 45 minutes, during which the temperature of the dispersion is controlled, typically within a range of 30 °C to 45 °C, either through intermittent sonication or through the use of a cooling jacket, to prevent thermal degradation of sensitive constituents. As a result of this process, non-polar impurities preferentially migrate into the hexane phase, while the more polar boswellic acids remain largely in the insoluble fraction. Following ultrasonic treatment, the dispersion is subjected to phase separation through centrifugation, wherein the mixture is transferred to a centrifuge system and rotated at speeds between 3,000 and 8,000 rpm for 10 to 25 minutes. This operation results in the formation of a clear supernatant containing dissolved terpenoids and a compact sediment comprising the enriched boswellic acid fraction.

The supernatant is decanted or siphoned off, and the remaining insoluble fraction is collected and subjected to further purification through re-extraction using ethyl acetate, a moderately polar solvent capable of selectively solubilizing boswellic acids. The insoluble fraction is transferred to a reflux extraction vessel, and ethyl acetate is added at a suitable solvent-to-solid ratio, typically between 1:4 and 1:10 (w/v). The mixture is heated under reflux conditions at a temperature between 60 °C and 75 °C for a duration of 1 to 3 hours, with continuous stirring to maintain uniform contact between solvent and extract. The reflux setup includes a condenser to prevent solvent loss and maintain a constant extraction volume.

During this re-extraction stage, boswellic acids, including 3-acetyl-11-keto-β-boswellic acid, are selectively dissolved into the ethyl acetate phase, leaving behind residual insoluble impurities. The resulting solution is then subjected to filtration to remove any undissolved material, followed by optional concentration to obtain a purified fraction enriched in boswellic acids. For example, in a representative implementation, 500 g of concentrated Boswellia extract is dispersed in 3 L of hexane and subjected to ultrasonic treatment at 30 kHz and 200 W/L for 30 minutes, followed by centrifugation at 5,000 rpm for 15 minutes. The recovered solid fraction is then re-extracted with 2.5 L of ethyl acetate under reflux at 65 °C for 2 hours, yielding a fraction with significantly increased boswellic acid concentration as confirmed by chromatographic analysis.

This sequential fractionation approach enables efficient removal of non-active terpenoid components while concentrating pharmacologically relevant boswellic acids, thereby improving the compositional quality of the extract and enhancing its suitability for downstream formulation. The combination of ultrasonic-assisted solvent interaction, centrifugal separation, and selective solvent re-extraction ensures reproducibility and scalability of the fractionation process while maintaining structural integrity of the active constituents.

In an embodiment, the chromatographic purification of the Commiphora extract (110)includes packing a silica column having particle size between 60 and 120 mesh and conditioning the column using hexane prior to sample loading, followed by gradient elution initiated with hexane: ethyl acetate ratio of 9:1 and gradually shifted to 7:3 over a volume range of 5 to 15 column volumes, wherein fractions are collected at intervals of 10 to 25 mL and monitored using thin-layer chromatography with a mobile phase comprising hexane: ethyl acetate (8: 2) and visualized under UV light at 254 nm, followed by pooling of fractions exhibiting retention factor values between 0.3 and 0.6 corresponding to guggulsterones.

In an embodiment, the chromatographic purification of the extract derived from Commiphora mukul is carried out using a controlled adsorption-based separation process employing a silica-packed column configured to selectively resolve guggulsterone isomers from co-extracted resinous constituents. The concentrated extract obtained after prior extraction and clarification is first optionally subjected to solvent exchange into a low-polarity medium such as hexane or a hexane-ethyl acetate mixture to ensure compatibility with the stationary phase and to prevent band broadening during loading. The chromatographic column is prepared by packing silica gel having a particle size distribution between 60 and 120 mesh into a vertically oriented cylindrical column using a wet-packing method, wherein the silica is slurried in hexane and introduced into the column under gentle vibration or tapping to eliminate air voids and achieve uniform bed density. The packed column is then conditioned by passing several column volumes of hexane through the bed to stabilize the packing structure and establish a baseline non-polar environment.

The prepared Commiphora extract is dissolved in a minimal volume of hexane or a weak hexane -ethyl acetate mixture and introduced onto the top of the column in a controlled manner to form a narrow loading band, thereby improving resolution. Elution is initiated using a solvent system comprising hexane and ethyl acetate at a ratio of 9:1, which favors the movement of highly non-polar constituents while retaining moderately polar compounds such as guggulsterones on the silica surface through adsorption interactions. The elution profile is then progressively modified by increasing the proportion of ethyl acetate in a gradient manner, typically transitioning from 9:1 to 7:3 over a total eluent volume corresponding to approximately 5 to 15 column volumes. This gradient modulation gradually increases the polarity of the mobile phase, weakening the interaction between guggulsterones and the silica surface and enabling their controlled elution without co-elution of more polar impurities.

Fractions are collected sequentially at defined intervals, typically between 10 and 25 mL per fraction, using a fraction collector synchronized with the eluent flow rate, which is generally maintained between 1 and 5 mL per minute depending on column dimensions. Each collected fraction is subjected to analytical monitoring using thin-layer chromatography, wherein a small aliquot of the fraction is spotted onto a silica-coated TLC plate and developed using a mobile phase comprising hexane and ethyl acetate in an 8:2 ratio. The developed plate is then visualized under ultraviolet light at 254 nm, allowing detection of UV-active components, including guggulsterones, which exhibit characteristic migration behavior. Fractions exhibiting retention factor values between 0.3 and 0.6 are identified as containing the desired E-and Z-guggulsterone isomers and are selectively pooled to form a purified fraction.

For example, in a representative implementation, 200 g of concentrated Commiphora extract is dissolved in 500 mL of hexane and loaded onto a silica column packed with 3 kg of 100-mesh silica. Elution is initiated with hexane: ethyl acetate (9:1) at a flow rate of 2 mL/min and gradually shifted to 7:3 over 10 column volumes. Fractions of 20 mL are collected and analyzed via TLC, resulting in identification of guggulsterone-containing fractions between fraction numbers 35 and 60. These fractions are pooled and concentrated to yield a purified extract with significantly enhanced guggulsterone content and reduced levels of non-target resin components.

The controlled gradient elution, combined with precise fraction monitoring and pooling based on retention characteristics, ensures effective separation of structurally similar compounds while maintaining high recovery of target molecules. The use of silica with defined particle size enhances surface area and adsorption consistency, while the gradual polarity shift in the mobile phase enables fine resolution of components with close physicochemical properties. This approach provides a reproducible and scalable purification strategy that improves downstream formulation performance by delivering a composition with well-defined chemical composition and reduced impurity burden.

In an embodiment, the combining of the Boswellia fraction and the Commiphora fraction is performed by initially dissolving each fraction separately in a co-solvent system comprising ethanol and propylene glycol in a volumetric ratio between 1:1 and 3:1, followed by gradual addition of a Commiphora solution into a Boswellia solution at a controlled rate of 1 to 5 mL per minute under continuous stirring at 200 to 500 rpm to obtain a mixture, while maintaining a temperature of the mixture between 30 °C and 40 °C to prevent localized precipitation and ensure molecular-level dispersion of active constituents.

In an embodiment, the Boswellia fraction and the Commiphora fraction are combined through a controlled solvent-mediated integration process configured to maintain physicochemical compatibility between triterpenoid-rich and steroidal resin fractions during formulation development. The Boswellia fraction, containing boswellic acid derivatives including acetylated keto-boswellic acids, exhibits comparatively higher resin viscosity and lower aqueous compatibility, whereas the Commiphora fraction enriched with guggulsterones possesses comparatively different polarity and solvation behavior. Direct mixing of such fractions under uncontrolled conditions may result in resin aggregation, localized supersaturation, precipitation of active constituents, and formation of non-uniform particulate domains that adversely affect downstream homogenization and storage stability. To address these issues, each fraction is independently dissolved in a co-solvent system comprising ethanol and propylene glycol prior to intermixing, thereby enabling gradual polarity adjustment and controlled molecular dispersion of phytochemical constituents.

In one implementation, the Boswellia fraction is introduced into a stainless-steel mixing vessel equipped with a temperature-regulated jacket and overhead mechanical stirrer, followed by addition of a pre-mixed ethanol-propylene glycol solvent system maintained at approximately 35 °C. The fraction is stirred continuously until a substantially clear or uniformly translucent solution is obtained without visible resin agglomerates. Separately, the Commiphora fraction is dissolved in a corresponding co-solvent system under similar thermal conditions to form a free-flowing solution with reduced viscosity. The solvent ratio is selected within the defined range to balance solubilization efficiency and viscosity reduction while minimizing excessive volatility associated with ethanol-rich systems. In an exemplary preparation, approximately 100 g of Boswellia fraction is dissolved in 250 mL of ethanol-propylene glycol mixture at a volumetric ratio of about 2:1, while approximately 60 g of Commiphora fraction is dissolved in 150 mL of the same solvent system under moderate agitation.

Following complete dissolution, the Commiphora solution is gradually introduced into the Boswellia solution through a controlled metered addition arrangement configured to maintain uniform concentration gradients within the mixture. Incremental addition at rates between 1 and 5 mL per minute prevents sudden local increases in hydrophobic constituent concentration that could otherwise trigger precipitation or phase separation. Continuous stirring within the specified rotational range generates sufficient convective flow to uniformly distribute incoming phytochemical molecules throughout the bulk medium without inducing excessive vortex formation or solvent aeration. Maintenance of the blending temperature between 30 °C and 40 °C further stabilizes solvent polarity and resin solubility while preventing thermal degradation of sensitive ketonic and acetylated constituents. The selected temperature range also assists in maintaining reduced viscosity of the resin fractions, thereby improving diffusion kinetics and intermolecular interaction during the blending operation.

During progressive addition, the solvent environment facilitates intermolecular association between boswellic acid derivatives and guggulsterone compounds at a dispersed molecular scale, resulting in formation of a substantially homogeneous phytochemical matrix suitable for subsequent homogenization or encapsulation procedures. The controlled blending operation minimizes formation of insoluble particulate clusters and reduces sedimentation tendencies commonly associated with resin-based herbal compositions. Optical analysis of the resulting mixture demonstrated absence of visible phase discontinuities and significantly lower particulate scattering relative to conventionally mixed preparations. Furthermore, accelerated storage evaluation conducted at elevated temperature conditions showed that compositions prepared according to the disclosed process retained uniform dispersion characteristics with minimal precipitation over extended storage intervals, whereas rapidly mixed comparative samples exhibited resin deposition and viscosity fluctuations within a short duration.

The disclosed blending methodology additionally improves reproducibility of active constituent distribution throughout the composition, thereby enabling more consistent downstream processing and dosage uniformity. Chromatographic evaluation performed on multiple samples collected from different regions of the prepared mixture demonstrated substantially consistent concentrations of boswellic acids and guggulsterones across the entire batch volume, indicating efficient molecular-level dispersion achieved through the controlled solvent-assisted integration process. Such uniformity contributes to enhanced formulation reliability, improved stability during storage and transport, and increased efficiency during subsequent emulsification, vesicle formation, spray drying, or microencapsulation operations.

In an embodiment, the high-shear homogenization includes subjecting combined bioactive blend and carrier medium to a two-stage shear process comprising a primary homogenization at 8,000-12,000 rpm for 5 -15 minutes followed by high-pressure homogenization at a pressure between 500 and 1,500 bar for 3 to 10 cycles, wherein each cycle includes passage through a homogenization valve with a gap size between 50 and 200 micrometers to produce a uniform dispersion with droplet size below 200 nm.

In an embodiment, the combined bioactive blend comprising dispersed Boswellia-derived triterpenoid constituents and Commiphora-derived steroidal fractions is subjected to a sequential high-energy homogenization procedure configured to reduce dispersed phase dimensions into the nanometric range while simultaneously improving compositional uniformity and colloidal stability of the phytotherapeutic system. Resin-derived botanical fractions typically exhibit heterogeneous particle distribution, high interfacial tension, and poor aqueous dispersibility when incorporated into carrier media, resulting in sedimentation, phase separation, and inconsistent bioactive release profiles. To overcome these limitations, the formulation is processed through a two-stage shear treatment involving an initial rotor-stator homogenization stage followed by high-pressure homogenization under controlled fluid compression conditions.

Initially, the combined bioactive blend and carrier medium are introduced into a primary homogenization chamber equipped with a high-speed rotor-stator assembly configured to generate intense localized shear gradients. During this stage, coarse resin agglomerates and viscous clusters formed during blending are mechanically disrupted into smaller dispersed domains through simultaneous shear, cavitation, and turbulent flow interactions. In one implementation, approximately 1 liter of phytotherapeutic dispersion containing Boswellia and Commiphora fractions is subjected to rotor-stator homogenization at approximately 10,000 rpm for about 10 minutes while maintaining the processing temperature below 40 °C through external cooling circulation. The selected rotational range provides sufficient kinetic energy to break down large droplets and resin particulates without inducing excessive thermal stress capable of degrading thermolabile phytoconstituents such as acetylated boswellic acids or guggulsterones. The resulting pre-homogenized mixture exhibits significantly reduced viscosity gradients and improved macroscopic uniformity suitable for downstream nanoscale processing.

Following primary homogenization, the dispersion is transferred into a high-pressure homogenization system configured to subject the mixture to repeated compression-decompression cycles through a precision homogenization valve. During each cycle, the formulation is forced at elevated pressure through a micro-gap region having a controlled gap dimension between approximately 50 and 200 micrometers. Passage through the restricted valve geometry generates extreme velocity differentials, cavitational collapse, impact collision, and intense fluid shear forces capable of fragmenting dispersed droplets into nanoscale structures. In one exemplary process, the dispersion is processed at approximately 1,000 bar for six homogenization cycles through a valve gap of approximately 100 micrometers, resulting in formation of a stable colloidal system having an average droplet diameter below 180 nm. Repeated cycling progressively narrows particle size distribution and minimizes occurrence of oversized aggregates that may otherwise destabilize the formulation during storage.

The reduction of droplet size into the sub-200 nm range substantially increases interfacial surface area available for interaction between the active phytoconstituents and the surrounding carrier environment. This enhanced surface exposure improves wettability and dissolution kinetics of hydrophobic boswellic acid derivatives and guggulsterones in aqueous physiological conditions. Dynamic light scattering analysis performed on the processed dispersion demonstrated a narrow polydispersity index indicative of uniform nanoscale distribution throughout the formulation. Microscopic evaluation further confirmed absence of large resin aggregates and demonstrated stable nanosized droplets dispersed uniformly within the carrier matrix. Such nanoscale dispersion contributes to improved physical stability by reducing gravitational sedimentation and minimizing coalescence tendencies during prolonged storage.

The sequential homogenization process additionally facilitates improved incorporation of lipidic or amphiphilic carrier materials surrounding the dispersed phytoconstituents, thereby enhancing entrapment efficiency and reducing leakage of active molecules during subsequent processing operations such as spray drying or microencapsulation. In comparative dissolution testing, homogenized nanoscale formulations prepared according to the disclosed process demonstrated substantially higher release of boswellic acids and guggulsterones within simulated gastrointestinal media relative to conventionally stirred suspensions. Pharmacokinetic evaluation further indicated increased systemic availability attributable to enhanced dispersion and reduced particle size. The homogenized compositions also exhibited improved storage robustness, maintaining consistent droplet distribution with negligible phase separation following accelerated stability exposure at elevated temperature and humidity conditions. Consequently, the disclosed two-stage shear process enables preparation of highly stable and reproducible phytotherapeutic dispersions possessing improved dispersibility, enhanced bioactive accessibility, and superior formulation consistency suitable for oral, topical, or encapsulated dosage applications.

In an embodiment, the carrier medium further includes incorporation of phosphatidylcholine in a concentration between 2% and 10% w/w, wherein the phosphatidylcholine is pre-dissolved in ethanol and added to the bioactive blend prior to homogenization, followed by controlled hydration using purified water added dropwise at a rate of 0.5 to 2 mL per minute under stirring at 300 to 700 rpm to facilitate formation of lipid-based vesicular structures encapsulating active constituents.

In an embodiment, the carrier medium is modified through incorporation of phosphatidylcholine to facilitate formation of lipid-organized vesicular assemblies capable of entrapping hydrophobic Boswellia and Commiphora bioactive constituents within amphiphilic membrane structures. Boswellic acids and guggulsterones inherently exhibit limited aqueous solubility and reduced permeability across biological membranes when administered in unstructured resin form, thereby restricting absorption efficiency and uniform bioactive delivery. The inclusion of phosphatidylcholine within the carrier medium enables development of a lipid-associated delivery environment in which the hydrophobic regions of the phospholipid interact with resin-derived active constituents while the hydrophilic head groups orient toward the surrounding aqueous phase, thereby promoting formation of stable vesicular structures capable of improving dispersion, protection, and transport of the phytochemical components.

In one implementation, phosphatidylcholine is initially dissolved in ethanol under moderate thermal conditions to obtain a substantially clear lipid solution prior to incorporation into the bioactive blend. Dissolution in ethanol assists in reducing lipid aggregation and enables uniform distribution of phospholipid molecules throughout the system before hydration-induced vesicle formation occurs. In an exemplary preparation, phosphatidylcholine is incorporated at approximately 5% w/w relative to the total composition and dissolved in ethanol maintained at approximately 35 °C under continuous stirring until complete solubilization is achieved. The phospholipid solution is subsequently combined with the previously prepared Boswellia-Commiphora bioactive blend under controlled agitation to produce a homogeneous lipid-active intermediate phase suitable for vesicle generation.

Following integration of the phospholipid and bioactive fractions, purified water is introduced gradually into the mixture through a controlled dropwise hydration operation while maintaining continuous stirring within the defined rotational range. Controlled aqueous addition induces progressive polarity transition within the medium, thereby initiating spontaneous self-assembly of phospholipid molecules into bilayered vesicular structures surrounding dispersed phytoconstituents. Gradual hydration prevents abrupt destabilization of the lipid phase and minimizes formation of irregular aggregates or precipitated resin clusters commonly observed during rapid aqueous dilution. In one representative process, purified water is added at a rate of approximately 1 mL per minute under stirring at about 500 rpm over a hydration interval of approximately 45 minutes. The selected agitation range generates sufficient mixing energy to uniformly distribute incoming aqueous droplets throughout the lipid-rich environment while avoiding excessive shear capable of disrupting forming vesicular assemblies.

As hydration progresses, phosphatidylcholine molecules reorganize into multilamellar and unilamellar vesicles in which hydrophobic Boswellia and Commiphora constituents become entrapped within lipid bilayers or internal hydrophobic domains. Microscopic and particle characterization analysis performed on the resulting formulation demonstrated formation of substantially spherical vesicular structures having nanoscale to submicron dimensions with uniform dispersion throughout the carrier matrix. Encapsulation of the active constituents within phospholipid domains provides a protective microenvironment that reduces direct exposure of oxidation-sensitive compounds to atmospheric oxygen, moisture, and external destabilizing conditions. The lipid-based organization additionally minimizes crystallization or precipitation of boswellic acid fractions during storage and improves compatibility of the formulation with aqueous physiological environments.

The vesicular carrier system generated through the disclosed hydration process further enhances transport efficiency of the phytochemical constituents across biological membranes due to structural similarity between phospholipid vesicles and cellular membrane architectures. In vitro permeation evaluation demonstrated substantially improved diffusion of boswellic acid derivatives across membrane models relative to non-lipid suspensions. Furthermore, dissolution testing showed accelerated release and increased apparent solubility of hydrophobic phytoconstituents attributable to the increased interfacial area and amphiphilic stabilization provided by the phosphatidylcholine assemblies. Stability studies conducted under accelerated storage conditions revealed significantly lower phase separation and reduced sedimentation in vesicle-containing formulations compared to formulations lacking phospholipid incorporation.

The disclosed phospholipid-assisted vesicle formation process additionally improves process reproducibility and downstream formulation adaptability. The resulting vesicular dispersion exhibits enhanced compatibility with subsequent homogenization, spray drying, microencapsulation, and oral or topical dosage preparation procedures due to the uniform distribution and stabilized association of the active constituents within the carrier matrix. Analytical evaluation of multiple production batches demonstrated consistent encapsulation efficiency and substantially uniform concentration of boswellic acids and guggulsterones throughout the formulation, thereby contributing to reliable dosage consistency and prolonged compositional stability during storage and administration.

In an embodiment, the stabilized phytotherapeutic composition is subjected to a post-processing stabilization step comprising adjusting pH to a range between 5.5 and 6.8 using a buffering system comprising citric acid and sodium citrate, followed by addition of an antioxidant selected from tocopherol or ascorbyl palmitate in a concentration between 0.01% and 0.5% w/w, and subsequently performing deaeration under vacuum at 50 to 100 mbar for 10 to 20 minutes to remove entrapped air and enhance compositional stability.

In an embodiment, the stabilized phytotherapeutic composition obtained following homogenization and vesicular dispersion processing is subjected to a post-processing conditioning operation configured to preserve chemical integrity, maintain colloidal uniformity, and improve long-term storage robustness of the composition. Resin-derived phytochemical constituents such as boswellic acids and guggulsterones are susceptible to oxidative degradation, hydrolytic transformation, viscosity fluctuation, and destabilization caused by dissolved oxygen or entrapped air introduced during high-shear processing operations. Additionally, fluctuations in pH may alter ionization behavior of acidic phytoconstituents and destabilize lipid-associated carrier structures, thereby affecting bioactive retention and physical uniformity of the composition over time. To address these challenges, the formulation is subjected to sequential pH conditioning, antioxidant incorporation, and vacuum deaeration under controlled process conditions.

Initially, the pH of the processed phytotherapeutic composition is adjusted to a mildly acidic range using a citrate-based buffering system comprising citric acid and sodium citrate. The selected pH range between approximately 5.5 and 6.8 provides a chemically balanced environment capable of minimizing hydrolysis of acetylated boswellic acid derivatives while simultaneously preserving vesicular integrity and preventing destabilization of phospholipid-associated structures. In one implementation, the formulation is transferred into a jacketed conditioning vessel equipped with a low-shear anchor stirrer, and a pre-prepared citrate buffer solution is introduced gradually under continuous agitation until a target pH of approximately 6.2 is achieved. Controlled addition of the buffering agents prevents localized pH gradients that could otherwise induce precipitation of resin fractions or partial disruption of dispersed lipid assemblies. The buffering environment additionally contributes to maintaining ionic balance within the dispersion system, thereby reducing aggregation tendencies and improving physicochemical consistency during storage.

Following pH adjustment, an antioxidant selected from tocopherol or ascorbyl palmitate is incorporated into the conditioned formulation to inhibit oxidative degradation pathways associated with exposure of phytoconstituents and lipid structures to dissolved oxygen and free radical species. High-energy homogenization processes frequently introduce oxidative stress conditions due to increased surface area exposure and entrainment of microscopic air bubbles within the formulation matrix. Incorporation of lipid-compatible antioxidants provides protective stabilization of unsaturated lipid domains and oxidation-sensitive ketonic phytoconstituents present within the Boswellia and Commiphora fractions. In one exemplary preparation, tocopherol is incorporated at approximately 0.1% w/w under moderate agitation until uniformly distributed throughout the dispersion medium. The antioxidant molecules localize preferentially within lipid-associated regions and act to suppress peroxide formation, color degradation, and breakdown of active constituents during prolonged storage exposure.

Subsequent to antioxidant incorporation, the formulation undergoes deaeration under controlled vacuum conditions to eliminate dissolved and entrapped gaseous pockets generated during blending and homogenization operations. The processed composition is subjected to reduced pressure within a vacuum deaeration chamber maintained between approximately 50 and 100 mbar for a duration sufficient to promote expansion and release of microbubbles dispersed throughout the formulation matrix. In one implementation, the deaeration step is conducted at approximately 75 mbar for about 15 minutes while maintaining slow agitation to facilitate upward migration of entrapped air without destabilizing the dispersed vesicular structures. Removal of microscopic air inclusions minimizes oxidative exposure, suppresses foam formation, and reduces the possibility of localized instability zones within the formulation. Elimination of entrapped gases additionally improves visual clarity, reduces variability in apparent viscosity, and enhances uniformity during filling and packaging operations.

The sequential stabilization treatment significantly improves preservation of the physicochemical characteristics of the phytotherapeutic composition during accelerated and long-term storage conditions. Stability evaluation performed under elevated temperature and humidity conditions demonstrated substantially reduced peroxide formation, lower viscosity drift, and minimized phase separation in compositions processed according to the disclosed conditioning procedure relative to untreated comparative formulations. Chromatographic analysis further confirmed improved retention of boswellic acid and guggulsterone content following extended storage intervals, indicating reduced oxidative and hydrolytic degradation of active constituents. The conditioned formulations also exhibited improved packaging compatibility and reduced incidence of container headspace pressure fluctuations caused by residual dissolved gases.

The disclosed post-processing stabilization procedure further contributes to maintaining batch-to-batch reproducibility and uniform bioactive distribution throughout the final composition. By preserving vesicular integrity, minimizing oxidative decomposition, and stabilizing the dispersion environment, the process enables consistent downstream performance during spray drying, encapsulation, oral formulation, topical application, or sustained-release dosage preparation. The resulting phytotherapeutic composition therefore exhibits improved storage endurance, enhanced compositional reliability, and sustained delivery performance suitable for pharmaceutical, nutraceutical, and therapeutic applications involving resin-derived botanical actives.

In an embodiment, the process further includes a controlled drying step applied to the stabilized phytotherapeutic composition by spray drying using an inlet temperature between 120 °C and 160 °C and an outlet temperature between 60 °C and 90 °C, wherein a feed rate is maintained between 3 and 10 mL/min and atomization is performed using a nozzle pressure between 1.5 and 3 bar to obtain a free-flowing powder with moisture content below 5% w/w.

In an embodiment, the stabilized phytotherapeutic composition is converted into a dry particulate formulation through a controlled spray-drying operation configured to preserve phytochemical integrity while improving storage stability, handling characteristics, and dosage adaptability of the composition. Resin-derived Boswellia and Commiphora fractions generally possess high stickiness, hygroscopic behavior, and poor flow properties in semi-solid form, which can create difficulties during packaging, transportation, encapsulation, and incorporation into oral dosage systems. Additionally, residual moisture present within liquid or semi-liquid phytotherapeutic dispersions may accelerate hydrolytic degradation, microbial susceptibility, and instability of vesicular carrier structures during prolonged storage. To overcome these limitations, the stabilized composition is subjected to a rapid solvent-removal process under carefully regulated thermal and atomization conditions capable of producing a substantially dry, uniform, and free-flowing powder while minimizing degradation of thermally sensitive active constituents.

Initially, the stabilized phytotherapeutic dispersion is introduced into a spray-drying assembly comprising a feed reservoir, atomization nozzle, heated drying chamber, and cyclone-based powder recovery system. Prior to drying, the formulation may optionally be filtered through a coarse filtration arrangement to remove oversized particulates capable of obstructing the atomization nozzle. The feed composition is maintained under continuous low-speed agitation within the reservoir to preserve homogeneity of dispersed lipidic and phytochemical components throughout the drying operation. In one implementation, the dispersion comprising vesicle-associated boswellic acids and guggulsterones is delivered into the spray dryer at a feed rate of approximately 6 mL/min while maintaining the inlet chamber temperature at approximately 140 °C and the outlet temperature near 75 °C. The selected inlet temperature range provides sufficient thermal energy for rapid evaporation of volatile solvent components immediately following atomization, whereas the comparatively lower outlet temperature limits prolonged exposure of the dried particles to elevated thermal conditions, thereby reducing degradation of oxidation-sensitive and acetylated phytoconstituents.

Atomization of the feed composition is performed through a pressure-assisted nozzle arrangement operated within the specified pressure range to generate fine droplets possessing substantially uniform dimensions. In one representative process, atomization is conducted at approximately 2 bar pressure to produce micron-scale droplets that rapidly undergo solvent evaporation upon contact with the heated drying medium. Controlled atomization pressure enables generation of droplets with sufficient surface area for efficient drying while minimizing formation of oversized wet particles or excessively fine dust fractions that could reduce product recovery efficiency. Rapid evaporation occurring during droplet transit through the drying chamber induces formation of solidified microstructures encapsulating the dispersed phytochemical and lipidic components within a dry particulate matrix. The short residence time of droplets within the heated environment substantially limits thermal decomposition despite the elevated inlet air temperature, thereby preserving the biological activity and structural integrity of the active constituents.

The resulting spray-dried particles exhibit reduced residual moisture content, typically below 5% w/w, thereby substantially improving resistance to hydrolytic degradation, microbial growth, and agglomeration during storage. Moisture reduction additionally minimizes stickiness commonly associated with resin-rich botanical formulations and enables formation of a free-flowing powder suitable for automated dosage manufacturing operations. Scanning electron microscopy performed on representative dried samples revealed substantially spherical or partially collapsed microstructures with smooth external morphology and limited fusion between adjacent particles, indicating efficient solvent removal and preservation of particle individuality during drying. Particle size analysis further demonstrated narrow distribution profiles contributing to improved flow consistency and reduced segregation during packaging or blending with excipients.

The spray-drying process additionally contributes to stabilization of the vesicular or nano-dispersed structures formed during preceding homogenization and phospholipid incorporation stages. Rapid immobilization of dispersed constituents within the dry particulate matrix reduces mobility of the active compounds and minimizes coalescence or aggregation that could otherwise occur during prolonged liquid-phase storage. Reconstitution studies demonstrated that the spray-dried powder rapidly redispersed in aqueous media to regenerate nanoscale dispersions exhibiting particle characteristics substantially similar to the pre-dried composition. This reconstitution capability enables convenient preparation of oral suspensions, sachet formulations, dispersible powders, capsules, and tablet dosage forms without substantial loss of dispersion quality.

Comparative stability studies conducted under accelerated environmental conditions showed that spray-dried formulations prepared according to the disclosed process retained significantly higher levels of boswellic acids and guggulsterones relative to non-dried liquid formulations stored under similar conditions. The dried formulations additionally exhibited improved handling efficiency, reduced transportation weight, enhanced packaging convenience, and extended shelf-life suitability for pharmaceutical and nutraceutical applications. Consequently, the controlled spray-drying operation enables conversion of the stabilized phytotherapeutic composition into a highly stable, process-compatible, and commercially adaptable dry formulation while preserving bioactive functionality and compositional uniformity.

In an embodiment, the process includes a microencapsulation step comprising dispersing the stabilized phytotherapeutic composition in an aqueous solution containing a polymer selected from poly(lactic-co-glycolic acid) or sodium alginate at a concentration between 1% and 5% w/v, followed by emulsification using a surfactant at 0.5% to 2% w/v and subsequent crosslinking using calcium chloride solution at 0.1 to 0.5 M concentration under continuous stirring at 300 to 800 rpm for 20 to 60 minutes to form encapsulated particles with controlled release characteristics.

In an embodiment, the stabilized phytotherapeutic composition is further processed through a microencapsulation operation configured to entrap Boswellia-derived and Commiphora-derived active constituents within a polymeric matrix capable of providing controlled release, enhanced physicochemical protection, and improved formulation stability during storage and administration. Resin-derived phytoconstituents such as boswellic acids and guggulsterones are susceptible to premature degradation, rapid release, and reduced gastrointestinal retention when administered in unprotected free form. Additionally, direct exposure of the active constituents to acidic gastric conditions or environmental oxygen may reduce bioactive availability and compromise long-term efficacy. To address these limitations, the phytotherapeutic composition is dispersed within a polymer-containing aqueous system and transformed into encapsulated particulate structures capable of modulating diffusion behavior and protecting the entrapped actives from destabilizing external conditions.

Initially, the stabilized phytotherapeutic composition is introduced into an aqueous polymer solution containing a biodegradable or ionically crosslinkable polymer selected from poly(lactic-co-glycolic acid) or sodium alginate. The polymer concentration is maintained within the specified range to achieve sufficient matrix-forming capability while preserving manageable viscosity for emulsification and particle formation. In one implementation, sodium alginate is dispersed in purified water at approximately 3% w/v under continuous agitation until a homogeneous viscous solution is obtained. The stabilized phytotherapeutic dispersion containing vesicle-associated boswellic acids and guggulsterones is subsequently introduced into the polymer solution under moderate stirring to generate a substantially uniform polymer-active mixture. Alternatively, in embodiments utilizing poly(lactic-co-glycolic acid), the polymer may first be dissolved within a volatile organic phase prior to emulsification with the phytotherapeutic composition to facilitate solvent-evaporation-based microcapsule formation.

Following preparation of the polymer-active mixture, emulsification is performed using a surfactant system maintained within the defined concentration range to facilitate generation of uniformly dispersed droplets suitable for encapsulation. The surfactant reduces interfacial tension between the polymer phase and surrounding medium, thereby improving droplet stabilization and preventing coalescence during particle formation. In one exemplary process, a non-ionic surfactant at approximately 1% w/v is incorporated into the mixture while agitation is maintained at approximately 500 rpm to generate fine dispersed microdomains containing entrapped phytochemical constituents. Controlled emulsification conditions contribute to formation of substantially uniform precursor droplets and minimize generation of oversized aggregates that could adversely affect release consistency and encapsulation efficiency.

Subsequent to emulsification, the dispersed system is subjected to a crosslinking operation through addition of calcium chloride solution under continuous stirring. In embodiments utilizing sodium alginate, calcium ions interact ionically with guluronic acid segments of the alginate polymer chains, thereby inducing formation of a three-dimensional hydrogel network surrounding the phytotherapeutic microdomains. In one representative implementation, calcium chloride solution having a concentration of approximately 0.25 M is added gradually into the emulsified dispersion while maintaining agitation at approximately 600 rpm for about 40 minutes. Controlled crosslinking under continuous mixing enables uniform diffusion of calcium ions throughout the polymer matrix and promotes formation of structurally consistent encapsulated particles with reduced aggregation. The duration of crosslinking is selected to ensure adequate hardening of the microcapsules without excessive densification that could impede subsequent release of the active constituents.

The resulting encapsulated particles comprise polymeric microstructures within which Boswellia and Commiphora bioactive constituents are entrapped in association with lipidic and polymeric domains. Microscopic examination of representative encapsulated particles demonstrated substantially spherical morphology with uniform structural integrity and limited surface rupture. Particle size distribution analysis revealed formation of microcapsules within a controlled dimensional range suitable for oral dosage, suspension, or sachet-based applications. Encapsulation efficiency measurements further demonstrated substantial retention of boswellic acid derivatives and guggulsterones within the polymer matrix, indicating effective incorporation of the phytotherapeutic constituents during the encapsulation process.

The polymeric microencapsulation arrangement significantly alters release kinetics of the active constituents by establishing a diffusion-regulated barrier surrounding the entrapped phytochemicals. In vitro release studies performed in simulated gastrointestinal conditions demonstrated gradual liberation of boswellic acids and guggulsterones over extended durations compared to non-encapsulated formulations, thereby reducing burst-release effects and improving sustained bioactive availability. The polymeric matrix additionally protects the active constituents from acidic degradation and oxidative exposure during transit through physiological environments. Encapsulated formulations exhibited improved retention of phytochemical content following accelerated stability exposure relative to non-encapsulated dispersions, thereby indicating enhanced preservation of bioactive integrity during storage.

The disclosed microencapsulation process further improves downstream formulation flexibility by enabling incorporation of the encapsulated particles into capsules, tablets, dry powders, suspensions, topical gels, and sustained-release delivery systems. The controlled-release behavior obtained through the polymeric barrier system contributes to prolonged residence time of the active constituents and reduced frequency of administration. Furthermore, the encapsulated structures exhibit improved handling characteristics, reduced stickiness associated with resin-derived extracts, and enhanced compatibility with automated pharmaceutical manufacturing operations. Consequently, the disclosed encapsulation methodology enables production of highly stable and release-regulated phytotherapeutic particles possessing improved storage endurance, enhanced delivery performance, and reproducible bioactive distribution suitable for pharmaceutical and nutraceutical applications involving Boswellia and Commiphora extracts.

In an embodiment, the Boswellia extract fraction obtained after ethyl acetate re-extraction is subjected to a controlled recrystallization step comprising dissolving the fraction in a mixed solvent system of ethyl acetate and methanol in a volumetric ratio between 3:1 and 1:1 at a temperature between 55 °C and 70 °C to form a clear solution, followed by gradual cooling at a controlled rate of 0.5 to 2 °C per minute to a temperature between 5 °C and 15 °C while maintaining continuous agitation at 100 to 300 rpm, and isolating crystalline precipitates enriched in 3-acetyl-11-keto-β-boswellic acid through vacuum filtration and drying under reduced pressure at 30-45 °C for 6 to 12 hours.

In an embodiment, the Boswellia extract fraction obtained following ethyl acetate re-extraction is subjected to a controlled recrystallization procedure configured to selectively enrich therapeutically significant boswellic acid constituents, particularly 3-acetyl-11-keto-β-boswellic acid, while simultaneously reducing co-extracted resinous impurities, amorphous fractions, pigments, and non-specific triterpenoid materials present within the partially purified extract. Conventional solvent extraction of Boswellia serrata resin frequently yields semi-solid fractions containing heterogeneous resin components with inconsistent purity profiles and variable crystallinity, which may adversely affect dissolution behavior, storage consistency, and reproducibility of downstream phytotherapeutic formulations. The disclosed recrystallization process therefore establishes a controlled solvent-mediated purification environment capable of generating highly enriched crystalline fractions exhibiting improved structural uniformity and compositional consistency.

Initially, the Boswellia extract fraction is transferred into a temperature-regulated dissolution vessel equipped with a mechanical agitation assembly and controlled heating arrangement. A mixed solvent system comprising ethyl acetate and methanol is introduced into the vessel within the defined volumetric ratio range to establish a solvent polarity environment suitable for selective dissolution of boswellic acid derivatives while limiting solubility of higher molecular weight resin impurities. In one implementation, approximately 100 g of the semi-purified Boswellia fraction is dispersed within a mixed solvent composition containing ethyl acetate and methanol in a ratio of approximately 2:1 and heated gradually to approximately 65 °C under continuous agitation at about 200 rpm. The elevated temperature facilitates disruption of intermolecular interactions between resin components and enables formation of a substantially clear homogeneous solution without visible undissolved particulates. The solvent combination is selected to achieve balanced solvation behavior, wherein boswellic acid derivatives remain fully dissolved at elevated temperature while less desirable resinous constituents exhibit comparatively lower solubility during subsequent cooling.

Following complete dissolution, the heated solution is subjected to a carefully regulated cooling sequence designed to promote controlled nucleation and growth of crystalline boswellic acid structures. Rapid temperature reduction is avoided because uncontrolled precipitation may entrap impurities within the developing crystal lattice and generate irregular amorphous aggregates with inconsistent purity. Accordingly, the temperature of the solution is reduced gradually at a controlled rate within the specified range while maintaining continuous agitation to preserve uniform thermal distribution throughout the crystallization medium. In one representative process, the solution temperature is reduced from approximately 65 °C to about 10 °C at a cooling rate of approximately 1 °C per minute under agitation maintained near 150 rpm. Controlled agitation during cooling prevents localized supersaturation zones and promotes uniform crystal nucleation throughout the solution volume, thereby improving crystal morphology and reducing aggregation.

As the temperature decreases, solubility of 3-acetyl-11-keto-β-boswellic acid progressively declines, resulting in formation of crystalline precipitates enriched in the target triterpenoid constituent. The gradual crystallization environment facilitates development of ordered crystal lattices with reduced incorporation of non-target resinous materials. Microscopic evaluation of the precipitated material demonstrated formation of substantially well-defined crystalline structures exhibiting improved uniformity relative to crude solvent-evaporated Boswellia extracts. Chromatographic analysis further indicated a substantial increase in the concentration of 3-acetyl-11-keto-β-boswellic acid following recrystallization, accompanied by a corresponding reduction in unidentified resin fractions and chromatographic impurity peaks.

Upon completion of crystallization, the precipitated crystals are separated from the mother liquor through vacuum-assisted filtration using a reduced-pressure filtration assembly configured to minimize thermal and oxidative stress during recovery. The isolated crystalline material is subsequently subjected to drying under reduced pressure at controlled temperature conditions to remove residual solvents while preserving structural integrity of the purified boswellic acid fraction. In one implementation, the recovered crystals are dried under vacuum at approximately 40 °C for about 8 hours until residual solvent content is substantially minimized. Controlled low-temperature drying prevents melting, discoloration, or thermal decomposition of the acetylated boswellic acid structures and preserves crystallinity of the isolated product.

The recrystallized Boswellia fraction obtained according to the disclosed process exhibits significantly improved physicochemical consistency and enhanced suitability for downstream phytotherapeutic formulation development. The increased purity and crystalline uniformity contribute to more reproducible dissolution behavior and improved batch-to-batch standardization during blending, homogenization, vesicle formation, and encapsulation operations. Comparative dissolution testing demonstrated that recrystallized fractions exhibited more consistent release kinetics relative to non-recrystallized resin fractions due to reduced amorphous impurity interference and improved particle uniformity. Stability evaluation further indicated reduced oxidative discoloration and improved storage endurance attributable to removal of unstable resinous impurities commonly associated with crude extracts.

In an embodiment, the Commiphora extract fraction, prior to chromatographic purification, is subjected to liquid-liquid partitioning by dispersing the concentrated extract in distilled water at a ratio of 1:2 to 1:6 (w/v), followed by sequential extraction using n-hexane and chloroform, each applied in two to four cycles with solvent-to-aqueous phase ratios between 1:1 and 3:1, wherein each extraction cycle includes agitation at 200 to 600 rpm for 15 to 30 minutes and phase separation by settling for 20 to 40 minutes, and pooling chloroform fractions for subsequent purification of guggulsterones.

In an embodiment, the Commiphora extract fraction obtained following primary solvent concentration is subjected to a sequential liquid-liquid partitioning procedure configured to selectively enrich steroidal guggulsterone constituents while removing non-polar waxes, fixed oils, chlorophyll derivatives, resinous ballast compounds, and other undesired co-extracted impurities prior to chromatographic purification. Crude Commiphora extracts derived from oleo-gum-resin sources typically contain a complex mixture of hydrophobic terpenoids, pigments, lipophilic resins, and steroidal ketones having overlapping solubility characteristics. Direct chromatographic purification of such heterogeneous extracts frequently results in reduced separation efficiency, column overloading, broad impurity tails, and inconsistent recovery of therapeutically significant guggulsterones. The disclosed liquid-liquid partitioning process therefore establishes a staged solvent-fractionation environment capable of progressively eliminating interfering constituents and concentrating the desired steroidal fraction before chromatographic refinement.

Initially, the concentrated Commiphora extract is dispersed in distilled water to generate an aqueous suspension suitable for polarity-based solvent partitioning. Hydration of the extract facilitates separation of water-compatible impurities from hydrophobic resin components and establishes a biphasic extraction environment for selective solvent transfer of target molecules. In one implementation, approximately 100 g of concentrated Commiphora extract is dispersed in about 400 mL of distilled water to form a substantially uniform suspension under moderate stirring. The aqueous dispersion may optionally be maintained at mildly elevated temperature conditions to reduce viscosity and improve mass transfer behavior during solvent extraction. Formation of the aqueous phase additionally assists in disrupting compact resin aggregates and exposes entrapped phytochemical constituents to the extraction solvents during subsequent partitioning operations.

Following preparation of the aqueous dispersion, the system is subjected to sequential extraction using n-hexane to selectively remove highly hydrophobic impurities including wax fractions, volatile oils, pigments, and resinous lipophilic contaminants. The n-hexane phase is introduced into the aqueous suspension within the defined solvent-to-aqueous phase ratio range and agitated under controlled mechanical mixing conditions to promote interfacial contact and mass transfer between phases. In one representative process, n-hexane is added at a ratio of approximately 2:1 relative to the aqueous phase and agitated at approximately 400 rpm for about 20 minutes. Controlled agitation provides sufficient mixing energy to increase solvent contact efficiency while minimizing formation of stable emulsions that could interfere with subsequent phase separation. During extraction, non-polar impurities preferentially migrate into the n-hexane phase due to their higher affinity for the hydrophobic solvent environment.

After agitation, the biphasic mixture is allowed to settle under quiescent conditions for a duration sufficient to enable distinct separation between the organic and aqueous layers. In one implementation, settling is maintained for approximately 30 minutes, resulting in formation of a clear upper n-hexane layer enriched with undesired hydrophobic ballast compounds and a lower aqueous phase retaining comparatively polar steroidal constituents including guggulsterones. The n-hexane layer is thereafter removed and discarded or separately processed, while the aqueous phase is retained for further extraction. Multiple extraction cycles may be performed sequentially to maximize removal of interfering impurities and improve purification efficiency prior to steroidal enrichment.

Subsequent to removal of the highly hydrophobic impurities, the aqueous phase is subjected to extraction using chloroform to selectively transfer guggulsterones and related steroidal ketones into the organic phase. Chloroform possesses solvent polarity characteristics suitable for extraction of steroidal bioactive molecules while leaving residual hydrophilic impurities within the aqueous phase. In one exemplary embodiment, chloroform is introduced at a solvent-to-aqueous ratio of approximately 2:1 and the mixture is agitated at approximately 500 rpm for about 25 minutes to facilitate diffusion of guggulsterones into the organic phase. The controlled extraction environment enhances partitioning efficiency while reducing co-transfer of undesirable aqueous contaminants. Following agitation, the mixture is permitted to settle until distinct phase separation is achieved, after which the chloroform layer enriched in guggulsterones is collected.

The chloroform extraction cycle may be repeated multiple times to maximize recovery of steroidal constituents from the aqueous phase. Pooling of the collected chloroform fractions produces an enriched intermediate extract exhibiting substantially increased concentration of guggulsterones and reduced levels of waxes, pigments, and resin contaminants relative to the starting Commiphora extract. Chromatographic evaluation of the pooled chloroform fraction demonstrated improved peak definition and reduced impurity interference compared to untreated crude extracts. The reduction in non-target resin load significantly improves downstream chromatographic separation efficiency by minimizing adsorption competition and reducing fouling of chromatographic stationary phases.

The disclosed partitioning methodology additionally improves reproducibility and standardization of the Commiphora-derived active fraction used in subsequent phytotherapeutic formulation operations. Removal of excess resinous ballast compounds contributes to improved clarity, reduced viscosity variation, and enhanced compatibility of the extract with homogenization, vesicular encapsulation, spray drying, and microencapsulation procedures. Stability studies further demonstrated reduced oxidative discoloration and lower sediment formation in formulations prepared using partition-purified Commiphora fractions compared to untreated extracts. The enriched steroidal fraction additionally exhibited improved consistency in guggulsterone concentration across multiple extraction batches, thereby enabling more reliable dosage standardization and improved formulation reproducibility for pharmaceutical and nutraceutical applications involving Commiphora-derived bioactive compounds.

In an embodiment, the filtration of the Boswellia and Commiphora extract fractions further includes a two-stage clarification procedure comprising an initial coarse filtration using a filter medium having pore size between 10 μm and 25 μm followed by fine filtration using a membrane filter having pore size between 0.2 μm and 0.45 μm, wherein the filtration is conducted under a pressure differential between 0.5 and 2 bar and maintained at a temperature between 25 °C and 35 °C to limit viscosity-induced flow resistance.

In an embodiment, the clarification of extract fractions obtained from Boswellia serrata and Commiphora mukul is performed through a sequential two-stage filtration procedure configured to remove suspended particulates, resinous debris, and micro-scale impurities while preserving dissolved bioactive constituents within the liquid phase. The extract fractions, following solvent extraction and prior to concentration or further purification, are transferred into a filtration assembly comprising a pressure-controlled housing capable of accommodating multiple filter media arranged in series. The filtration process is conducted under a controlled pressure differential ranging from 0.5 to 2 bar, generated using a pump or pressurized gas system, which ensures consistent flow through the filter layers without causing structural disruption of the filter media or forcing undesirable particulates through the pores.

The first stage of filtration involves passage of the extract through a coarse filter medium having a pore size between 10 μm and 25 μm. This coarse filtration stage is designed to remove larger suspended solids, including undissolved resin fragments, fibrous plant material, and aggregated impurities that may have formed during extraction or cooling. The filter medium may be composed of materials such as cellulose, polypropylene, or sintered metal, and is selected based on chemical compatibility with the solvent system and mechanical durability under pressure. The extract is introduced into the filtration unit at a controlled flow rate, and the pressure differential is adjusted within the specified range to maintain steady throughput while preventing clogging or channeling within the filter bed. This stage significantly reduces turbidity and prepares the extract for subsequent fine filtration.

Following coarse clarification, the partially clarified extract is directed into a second filtration stage employing a fine membrane filter with a pore size between 0.2 μm and 0.45 μm. This membrane filtration stage is critical for removal of fine particulates, colloidal impurities, and microbial contaminants, thereby producing a clear and refined extract suitable for downstream processing such as concentration, fractionation, or chromatographic purification. The membrane filter is typically composed of polymeric materials such as polyethersulfone or polytetrafluoroethylene, which provide high chemical resistance and precise pore size control. The filtration is conducted under the same controlled pressure range, ensuring that the membrane integrity is maintained while achieving efficient separation of sub-micron particles.

Throughout both filtration stages, the temperature of the extract is maintained within a range of 25 °C to 35 °C using a temperature-controlled environment or jacketed filtration system. This temperature range is selected to balance fluid viscosity and solubility characteristics of the extract; maintaining the extract within this range prevents excessive viscosity that could impede flow through the filters while also avoiding elevated temperatures that could lead to degradation or volatilization of sensitive compounds. The controlled temperature also ensures that dissolved bioactive constituents remain in solution and are not inadvertently precipitated during filtration.

For example, in a representative implementation, 10 liters of Boswellia extract obtained after solvent extraction is first passed through a 20 μm polypropylene filter at a pressure of 1 bar, resulting in removal of visible particulates and reduction in turbidity. The filtrate is then subjected to membrane filtration using a 0.45 μm polyethersulfone membrane under a pressure of 1.5 bar at 30 °C, producing a clear extract with minimal suspended solids. A similar procedure is applied to Commiphora extract, ensuring consistent clarity and purity across both fractions. Analytical measurements of turbidity and particulate content before and after filtration demonstrate a substantial reduction in suspended matter, while chemical analysis confirms that concentrations of boswellic acids and guggulsterones remain unaffected.

This two-stage clarification approach enables efficient removal of impurities across a broad size range while maintaining process continuity and minimizing loss of active constituents. The controlled pressure and temperature conditions ensure reproducible filtration performance, reduce the risk of filter fouling, and provide a clarified extract with improved suitability for subsequent high-precision purification and formulation steps.

In an embodiment, the concentration under reduced pressure further includes real-time monitoring of solvent removal by measuring condensate volume and correlating it with initial solvent input, and terminating the concentration step upon achieving a residual solvent content below 5% w/w, followed by immediate transfer of the semi-solid extract into an inert atmosphere chamber maintained with nitrogen or argon at oxygen levels below 2% to limit oxidative transformation of active constituents.

In an embodiment, the concentration stage performed under reduced pressure is further enhanced by incorporating a real-time monitoring and control mechanism that enables precise determination of solvent removal progression and ensures consistent attainment of a target residual solvent threshold in the extract derived from Boswellia serrata and Commiphora mukul. The extract, following filtration, is subjected to rotary evaporation within a sealed system equipped with a condensate collection assembly and measurement interface, wherein the volume of condensed solvent is continuously or periodically measured using calibrated volumetric sensors or graduated receivers. The initial solvent volume introduced during extraction is pre-recorded within the system control unit, and the measured condensate volume is dynamically correlated with this initial value to estimate the extent of solvent removal in real time.

As the evaporation proceeds under controlled pressure and temperature conditions, the system computes the remaining solvent fraction within the extract based on mass balance calculations, taking into account evaporation rates, condensate recovery efficiency, and potential solvent retention within the viscous matrix. The process is not terminated based on fixed time intervals but is instead governed by this real-time correlation, allowing the concentration step to continue until the residual solvent content is reduced below a predefined threshold, typically less than 5% w/w. This approach ensures that variations in batch composition, solvent composition, or operating conditions do not result in under-or over-processing, thereby improving consistency and reliability of the semi-solid extract.

Upon reaching the target residual solvent level, the concentrated extract is immediately transferred, either through a closed conduit or under sealed conditions, into an inert atmosphere chamber designed to minimize exposure to oxygen. The chamber is purged with an inert gas such as nitrogen or argon, and the internal environment is regulated to maintain oxygen concentration below 2%, typically achieved through continuous gas flow or controlled displacement of ambient air. The chamber may be equipped with oxygen sensors and flow control systems to ensure maintenance of the desired inert conditions throughout the transfer and holding period.

The immediate transfer into the inert environment prevents post-concentration exposure of the extract to atmospheric oxygen, which is particularly critical for preserving the structural integrity of oxidation-sensitive constituents such as boswellic acids and guggulsterones. The reduced oxygen environment limits oxidative reactions, discoloration, and formation of degradation by-products that could otherwise occur during handling or storage of the semi-solid extract. Additionally, the inert atmosphere assists in the removal of any residual volatile solvent traces by facilitating their diffusion out of the extract matrix without introducing oxidative stress.

For example, in a representative implementation, 8 liters of filtered extract are subjected to rotary evaporation, and approximately 7.5 liters of solvent are recovered as condensate. The system continuously tracks the condensate volume and determines that residual solvent content has reached approximately 4.5% w/w. At this point, the process is automatically terminated, and the concentrated extract is transferred via a sealed pipeline into a nitrogen-purged chamber maintained at oxygen levels of 1.5%. The extract is held under these conditions for a defined period, during which no significant change in composition or color is observed, indicating preservation of chemical stability.

This integration of real-time monitoring with controlled termination and inert atmosphere handling ensures that the concentration process yields a semi-solid extract with consistent solvent content and preserved chemical composition, while also minimizing variability and degradation risks associated with conventional evaporation and handling techniques.

In an embodiment, the combining of the Boswellia and Commiphora fractions further includes a pre-mixing equilibration step comprising holding the individual fractions at a temperature between 35 °C and 45 °C for 20 to 60 minutes to equalize viscosity, followed by introduction of a co-solubilizing agent selected from polyethylene glycol having molecular weight between 200 and 600 in a concentration between 5% and 20% w/w prior to blending, and performing mixing under a controlled shear rate between 100 s⁻¹ and 500 s⁻¹ to obtain a uniform intermediate mixture.

In an embodiment, the integration of the purified fractions obtained from Boswellia serrata and Commiphora mukul is preceded by a pre-mixing equilibration stage configured to normalize rheological properties of the individual fractions and thereby enable controlled blending without phase instability or localized concentration gradients. The Boswellia-derived fraction and the Commiphora-derived fraction, each obtained after respective purification steps, are independently transferred into temperature-controlled holding vessels equipped with gentle agitation capability. Each fraction is maintained at a temperature within the range of 35 °C to 45 °C for a duration between 20 and 60 minutes, during which the viscosity of the semi-solid or viscous liquid fractions decreases due to thermal softening and uniform heat distribution. This equilibration period ensures that both fractions attain comparable flow characteristics, thereby reducing shear imbalance and preventing uneven dispersion when brought into contact during blending.

Following viscosity equilibration, a co-solubilizing agent selected from polyethylene glycol having a molecular weight between 200 and 600 is introduced into one or both fractions prior to combining. The polyethylene glycol is added in a concentration ranging from 5% to 20% w/w relative to the total mass of the fractions and is typically pre-heated to a temperature compatible with the equilibration range to ensure miscibility. The addition of polyethylene glycol modifies the solvent environment by increasing polarity compatibility and enhancing hydrogen bonding interactions, thereby facilitating solubilization of both boswellic acids and guggulsterones within a common medium. This step reduces the likelihood of phase separation and improves the uniformity of the resulting mixture.

The blending operation is then carried out under controlled shear conditions using a mixing apparatus capable of delivering a defined shear rate between 100 s⁻¹ and 500 s⁻¹ . The fractions are combined gradually, either by simultaneous introduction into a mixing chamber or by controlled addition of one fraction into the other, while maintaining continuous agitation within the specified shear range. The selected shear rate is sufficient to promote interfacial mixing and dispersion of the fractions without generating excessive turbulence or heat that could destabilize the constituents. The temperature of the mixture is maintained within the equilibration range during this step to preserve viscosity balance and solubility conditions.

For example, in a representative implementation, 200 g of Boswellia fraction and 150 g of Commiphora fraction are each maintained at 40 °C for 30 minutes to achieve viscosity equilibration. Polyethylene glycol 400 is added at 10% w/w to the combined system, and the fractions are blended under a shear rate of approximately 250 s⁻¹ using a controlled-speed mixer. The resulting intermediate mixture exhibits a uniform, homogenous appearance without visible phase separation or particulate formation. Analytical sampling from different regions of the mixture confirms consistent distribution of active constituents, indicating effective blending.

This pre-mixing equilibration and co-solubilization approach enables improved compatibility between the fractions, ensuring that differences in viscosity and polarity do not hinder mixing efficiency. The controlled shear blending further ensures that the resulting intermediate mixture is stable and suitable for subsequent processing steps such as homogenization and carrier incorporation, while maintaining the integrity and distribution of the bioactive compounds.

In an embodiment, the homogenization step further includes temperature regulation by circulating a cooling medium through a jacketed vessel to maintain the composition temperature within a range of 20 °C to 30 °C during high-shear processing, and intermittently pausing homogenization cycles for 2 to 5 minutes after every 5 to 10 minutes of operation to prevent thermal degradation of thermolabile constituents.

In an embodiment, the homogenization stage applied to the combined bioactive blend derived from Boswellia serrata and Commiphora mukul is further refined by incorporating an active temperature regulation protocol designed to maintain thermal stability of the formulation during high-energy shear processing. The homogenization is conducted within a jacketed processing vessel equipped with an external circulation system through which a cooling medium, such as chilled water or a glycol-based coolant, is continuously passed. The circulation system is connected to a temperature control unit capable of maintaining the internal composition temperature within a narrow range of 20 °C to 30 °C despite the heat generated by mechanical shear forces. Temperature sensors positioned within the vessel provide real-time feedback to the control unit, enabling dynamic adjustment of coolant flow rate and temperature to ensure consistent thermal conditions throughout the homogenization process.

During high-shear processing, particularly in rotor-stator or high-pressure homogenization systems, mechanical energy is converted into heat due to frictional forces and turbulence within the fluid. If not properly controlled, this heat accumulation can elevate the temperature of the composition beyond levels that are compatible with the stability of thermolabile constituents such as boswellic acids and guggulsterones. To mitigate this, the process incorporates an intermittent operation protocol wherein homogenization is conducted in cycles, each lasting between 5 and 10 minutes, followed by a pause interval of 2 to 5 minutes. During these pause intervals, the agitation or pressure application is temporarily halted while the cooling system continues to operate, allowing dissipation of accumulated thermal energy and restoration of the composition temperature to within the desired range.

This cyclic homogenization approach ensures that the composition is exposed to sufficient shear energy to achieve particle size reduction and uniform dispersion, while preventing continuous heat buildup that could lead to degradation, structural modification, or volatilization of sensitive components. The pause intervals are optimized based on batch size, viscosity, and energy input, ensuring that the process remains efficient while maintaining compositional integrity.

For example, in a representative implementation, a bioactive mixture undergoing rotor-stator homogenization at 10,000 rpm is processed for 8-minute intervals followed by 3-minute cooling pauses, during which chilled water at 15 °C is circulated through the vessel jacket. Temperature monitoring indicates that the composition temperature rises to approximately 29 °C during active homogenization and is reduced back to approximately 23 °C during the pause interval, thereby maintaining the overall temperature within the specified range. After multiple cycles, the desired particle size reduction is achieved without observable changes in color, viscosity, or chemical composition, indicating preservation of the active constituents.

This integration of continuous cooling and intermittent operation provides a controlled thermal environment that supports effective homogenization while maintaining stability of the formulation. The approach ensures reproducibility across batches and compatibility with sensitive phytochemical systems, thereby enabling reliable production of a finely dispersed and stable phytotherapeutic composition.

In an embodiment, the carrier medium preparation includes pre-dissolving surfactants selected from polysorbate 80 or lecithin in a lipid phase comprising medium-chain triglycerides at a temperature between 40 °C and 60 °C, followed by gradual addition of an aqueous phase maintained at 25 °C to 35 °C under continuous stirring at 400 to 900 rpm to form a pre-emulsion prior to incorporation of the bioactive blend.

In an embodiment, the preparation of the carrier medium used for incorporation of the bioactive blend derived from Boswellia serrata and Commiphora mukul is carried out through a controlled pre-emulsification process designed to establish a stable biphasic interface prior to introduction of the active constituents. The carrier medium is initially formed by preparing a lipid phase comprising medium-chain triglycerides, which are selected due to their favorable solubilizing capacity for lipophilic compounds and their compatibility with oral and topical formulations. The lipid phase is transferred into a temperature-controlled vessel and heated to a temperature between 40 °C and 60 °C using a jacketed heating system, ensuring reduction in viscosity and enabling efficient dissolution of surfactants.

Surfactants selected from polysorbate 80 or lecithin are then introduced into the heated lipid phase in a controlled manner, typically under moderate stirring conditions. The surfactants are allowed to fully dissolve within the lipid medium, forming a homogeneous solution in which the amphiphilic molecules are uniformly distributed. The selection of surfactant type and concentration is based on the desired hydrophilic-lipophilic balance, enabling effective stabilization of subsequent emulsified structures. The elevated temperature ensures complete solubilization of the surfactant molecules and promotes alignment of their hydrophobic moieties with the lipid phase, thereby preparing the system for efficient emulsification.

Once the lipid-surfactant mixture is fully homogenized, an aqueous phase comprising purified water or a buffered solution is prepared separately and maintained at a temperature between 25 °C and 35 °C. The temperature differential between the lipid phase and aqueous phase is controlled to prevent sudden viscosity changes or phase inversion during mixing. The aqueous phase is then introduced gradually into the lipid phase under continuous stirring, wherein the agitation speed is maintained between 400 and 900 rpm using a mechanical stirrer or impeller system. The gradual addition is typically carried out using a controlled dosing mechanism to ensure uniform dispersion of the aqueous phase into the lipid medium, resulting in the formation of a coarse pre-emulsion.

During this process, the surfactant molecules orient at the interface between the lipid and aqueous phases, reducing interfacial tension and facilitating the formation of dispersed droplets. The controlled stirring ensures that droplet formation occurs uniformly throughout the medium, preventing coalescence and enabling the generation of a stable pre-emulsion with consistent droplet size distribution. The resulting system exhibits a milky or translucent appearance, indicating successful dispersion of the aqueous phase within the lipid matrix.

For example, in a representative implementation, 500 mL of medium-chain triglycerides is heated to 50 °C, and 50 g of polysorbate 80 is dissolved under stirring at 500 rpm until a clear solution is obtained. Separately, 300 mL of purified water is maintained at 30 °C and gradually introduced into the lipid phase over a period of 20 minutes while increasing the stirring speed to 700 rpm. The resulting mixture forms a stable pre-emulsion with uniform droplet distribution, which serves as a suitable base for subsequent incorporation of the bioactive blend.

This pre-emulsification step establishes a stable carrier environment that enhances compatibility with the bioactive constituents and facilitates efficient dispersion during subsequent homogenization. The controlled temperature conditions, surfactant dissolution, and gradual phase addition ensure formation of a uniform and stable intermediate system, thereby improving the consistency and performance of the final phytotherapeutic composition.

In an embodiment, the process further includes a particle size reduction step applied after homogenization, comprising passing the composition through a microfluidization chamber at pressures between 800 and 1,800 bar for 5 to 15 passes, wherein each pass involves acceleration through microchannels having dimensions between 50 and 150 micrometers to achieve a particle size distribution with a polydispersity index below 0.3.

In an embodiment, the process further incorporates an advanced particle size reduction stage applied subsequent to the primary homogenization of the bioactive blend derived from Boswellia serrata and Commiphora mukul, wherein the pre-homogenized dispersion is subjected to microfluidization under high-pressure conditions to achieve a refined nanoscale distribution with enhanced uniformity. The homogenized composition, typically exhibiting a coarse to intermediate droplet size following rotor-stator and high-pressure homogenization, is introduced into a microfluidization unit comprising a high-pressure pump and an interaction chamber containing microchannels engineered with precise geometries. The system is configured to operate at pressures ranging from 800 to 1,800 bar, enabling the generation of extremely high fluid velocities as the composition is forced through the microchannels.

Within the microfluidization chamber, the composition undergoes intense shear, impact, and cavitation effects as it passes through microchannels having characteristic dimensions between 50 and 150 micrometers. The acceleration of the fluid through these confined pathways results in collision of fluid streams and rapid pressure differentials, which act to disrupt residual aggregates and reduce droplet size to the nanometer scale. Each pass through the chamber contributes incrementally to the refinement of the particle size distribution, and the process is typically repeated for 5 to 15 passes depending on the initial droplet size, viscosity of the formulation, and desired final characteristics.

The process is conducted under controlled thermal conditions, wherein the temperature of the composition is monitored continuously and regulated through integrated cooling systems to prevent excessive heat buildup resulting from high-pressure operation. Between successive passes, the composition may be circulated through a heat exchanger or cooled reservoir to maintain the temperature within a range compatible with the stability of the active constituents. This ensures that the structural integrity of boswellic acids and guggulsterones is preserved while achieving the desired level of size reduction.

As a result of repeated microfluidization cycles, the composition attains a highly uniform particle size distribution characterized by a mean droplet size typically below 200 nm and a polydispersity index below 0.3, indicating narrow distribution and minimal variation between particles. The reduction in polydispersity is particularly significant, as it reflects improved uniformity and stability of the dispersion, reducing the likelihood of coalescence or sedimentation during storage.

For example, in a representative implementation, a pre-homogenized emulsion with an initial mean droplet size of approximately 250 nm is subjected to microfluidization at 1,200 bar for 10 passes through a chamber with 100 μm microchannels. After processing, particle size analysis using dynamic light scattering indicates a reduction in mean droplet size to approximately 120 nm with a polydispersity index of 0.25. The resulting dispersion exhibits enhanced stability, with no observable phase separation or aggregation over extended storage conditions.

This microfluidization step provides an additional level of refinement beyond conventional homogenization, enabling precise control over particle size and distribution. The high-energy interactions within the microchannels ensure thorough disruption of residual aggregates and promote uniform dispersion of the bioactive constituents within the carrier medium. The resulting nanoscale formulation demonstrates improved physical stability and compatibility with various delivery systems, thereby supporting consistent performance of the phytotherapeutic composition.

In an embodiment, the process further includes a controlled solvent removal step after blending and homogenization, comprising subjecting the composition to rotary evaporation at a temperature between 35 °C and 45 °C and pressure between 100 and 200 mbar for 20 to 60 minutes, followed by vacuum drying at 40-50 °C for an additional 2 to 6 hours to remove residual organic solvents to levels below 0.5% w/w.

In an embodiment, the process further incorporates a controlled solvent removal stage following blending and homogenization of the bioactive system derived from Boswellia serrata and Commiphora mukul, wherein residual organic solvents present from prior extraction and co-solvent blending are removed under carefully regulated thermal and pressure conditions to yield a composition with minimal solvent content while preserving dispersion integrity. The homogenized composition, typically comprising a lipid-surfactant carrier system containing dispersed bioactive constituents, is transferred into a rotary evaporation assembly equipped with a vacuum control system and a temperature-regulated bath. The evaporation is carried out at a temperature between 35 °C and 45 °C and under reduced pressure maintained within a range of 100 to 200 mbar, which lowers the boiling point of the solvent system and enables efficient removal of volatile components without exposing the composition to elevated thermal stress.

During rotary evaporation, the flask is rotated at a controlled speed, typically between 60 and 120 rpm, to form a thin film of the composition along the inner wall surface, thereby increasing the effective surface area for evaporation and promoting uniform solvent removal. The process is continued for a duration of 20 to 60 minutes, during which solvent vapors are continuously condensed and collected in a receiving vessel. The reduced pressure environment ensures that solvents such as ethanol or residual organic components are removed efficiently while maintaining the temperature below thresholds that could compromise the structural integrity of the dispersed bioactive compounds or destabilize the emulsion system.

Following the rotary evaporation phase, the partially dried composition is subjected to a secondary vacuum drying step to remove residual solvent traces that may remain entrapped within the viscous matrix or within nanoscale droplets of the dispersion. The composition is transferred into a vacuum drying chamber or tray dryer operating at a temperature between 40 °C and 50 °C under sustained vacuum conditions, typically below 100 mbar. This stage is maintained for an extended duration of 2 to 6 hours, allowing gradual diffusion and evaporation of residual solvent molecules from within the internal structure of the formulation. The drying environment may include gentle agitation or thin-layer spreading of the composition to facilitate uniform solvent removal and prevent localized accumulation.

The progression of solvent removal is monitored through periodic sampling and analysis using techniques such as gas chromatography or gravimetric measurement to determine residual solvent content. The process is continued until the solvent level is reduced to below 0.5% w/w, ensuring compliance with safety and stability requirements. The controlled combination of rotary evaporation and vacuum drying ensures that solvent removal occurs in a gradual and uniform manner, preventing abrupt changes in viscosity or phase behavior that could otherwise lead to aggregation or destabilization of the dispersed system.

For example, in a representative implementation, 1 liter of homogenized formulation containing approximately 10% residual ethanol is subjected to rotary evaporation at 40 °C and 150 mbar for 45 minutes, resulting in reduction of solvent content to approximately 2% w/w. The composition is then transferred to a vacuum dryer maintained at 45 °C for 4 hours, after which residual solvent analysis indicates levels below 0.3% w/w. The final product retains its nanoscale dispersion characteristics and exhibits no visible phase separation or degradation.

This controlled solvent removal protocol ensures that the final phytotherapeutic composition is substantially free of residual organic solvents while maintaining the structural and compositional integrity of the formulation. The staged removal approach allows efficient elimination of both free and entrapped solvent fractions, resulting in a stable and pharmaceutically acceptable product suitable for further processing or direct formulation into dosage forms.

In an embodiment, the process includes incorporation of a stabilizing polysaccharide selected from xanthan gum or hydroxypropyl methylcellulose in a concentration between 0.2% and 1.5% w/w, wherein the polysaccharide is hydrated in purified water for 30 to 90 minutes prior to addition, and subsequently introduced into the composition under stirring at 300 to 700 rpm to increase viscosity and maintain dispersion uniformity.

In an embodiment, the process further includes incorporation of a stabilizing polysaccharide into the formulated composition derived from Boswellia serrata and Commiphora mukul, wherein the polysaccharide functions to enhance viscosity, prevent phase separation, and maintain long-term uniformity of the dispersion. The stabilizing agent is selected from xanthan gum or hydroxypropyl methylcellulose, both of which exhibit high water-binding capacity and the ability to form structured networks within aqueous or semi-aqueous systems. The selected polysaccharide is first subjected to a controlled hydration step prior to its incorporation into the composition to ensure complete activation and to avoid formation of undissolved aggregates.

The hydration process is carried out by dispersing the polysaccharide in purified water under continuous stirring, typically at ambient or slightly elevated temperatures between 25 °C and 35 °C. The dispersion is maintained under agitation for a duration ranging from 30 to 90 minutes, during which the polymer chains progressively absorb water, swell, and form a viscous, homogeneous solution. The rate of hydration is controlled to prevent clumping, and in certain implementations, the polysaccharide may be gradually sprinkled into the water under vortex conditions to ensure uniform wetting of individual particles. The resulting hydrated solution exhibits a consistent viscosity profile and is free of lumps, thereby enabling effective incorporation into the main formulation.

Once hydration is complete, the polysaccharide solution is introduced into the previously homogenized bioactive composition under controlled stirring conditions. The addition is performed gradually, either through a metered dosing system or manual addition in small increments, while maintaining agitation within a range of 300 to 700 rpm. This controlled introduction ensures uniform distribution of the polysaccharide throughout the composition and prevents localized viscosity gradients or gel formation. The interaction between the hydrated polymer chains and the dispersed droplets within the formulation leads to an increase in bulk viscosity and formation of a structured network that stabilizes the dispersion against sedimentation, creaming, or coalescence.

The concentration of the polysaccharide is maintained within a range of 0.2% to 1.5% w/w relative to the total composition, which is sufficient to provide stabilization without excessively increasing viscosity to levels that could hinder processing or administration. The selected concentration range allows optimization of flow properties, ensuring that the final formulation remains pourable or processable while maintaining uniform distribution of bioactive constituents.

For example, in a representative implementation, 0.8% w/w xanthan gum is dispersed in purified water and hydrated under stirring at 500 rpm for 60 minutes to form a uniform viscous solution. This hydrated solution is then gradually introduced into 1 liter of homogenized bioactive composition under stirring at 600 rpm, resulting in a stable dispersion with increased viscosity and no observable phase separation over extended storage. Similarly, hydroxypropyl methylcellulose at 1% w/w can be hydrated and incorporated under similar conditions to achieve comparable stabilization effects.

The incorporation of the hydrated polysaccharide enhances the physical stability of the composition by reducing mobility of dispersed particles and improving resistance to gravitational separation. The resulting formulation exhibits consistent rheological behavior and maintains uniform distribution of boswellic acids and guggulsterones across the bulk medium, thereby ensuring reliability in subsequent processing and application.

In an embodiment, the process further includes a sterilization step comprising passing the final composition through a sterile filtration unit having a pore size of 0.22 μm under aseptic conditions, followed by filling into pre-sterilized containers in an environment maintained at ISO Class 5 cleanliness, wherein the entire operation is conducted at a temperature between 20 °C and 25 °C to maintain compositional stability.

In an embodiment, the process further includes a sterilization stage applied to the final phytotherapeutic composition derived from Boswellia serrata and Commiphora mukul, wherein microbial and particulate contamination is removed through a controlled sterile filtration procedure followed by aseptic filling under highly regulated environmental conditions. The fully processed composition, typically present as a stabilized dispersion or emulsion with nanoscale particle distribution, is transferred into a sterile filtration assembly comprising a membrane filter unit having a nominal pore size of 0.22 μm. The filtration unit is constructed from chemically compatible materials such as polyethersulfone or polyvinylidene fluoride and is pre-sterilized using standard sterilization methods such as steam sterilization or gamma irradiation prior to use.

The composition is passed through the membrane filter under aseptic conditions using a controlled pressure differential, typically generated by a peristaltic pump or pressurized inert gas, ensuring that the flow rate remains within limits that prevent disruption of the dispersion structure. The 0.22 μm pore size is selected to effectively remove bacteria and other microbial contaminants, as well as fine particulate matter, while allowing passage of the dissolved and nanoscale dispersed bioactive constituents. The filtration process is carried out in a closed system to prevent exposure to the external environment, and all fluid pathways are sterilized prior to operation.

Following sterile filtration, the composition is immediately transferred into a filling system located within a controlled cleanroom environment maintained at ISO Class 5 cleanliness. This environment is achieved through high-efficiency particulate air (HEPA) filtration systems that maintain extremely low levels of airborne particulates and microbial contamination. The filling operation involves dispensing the ctepilized composition into pre-sterilized containers, such as vials, bottles, or sachets, using automated or semi-automated filling equipment designed for aseptic processing. The containers themselves are pre-treated through sterilization processes such as autoclaving or dry heat sterilization and are introduced into the cleanroom through airlocks or transfer chambers to maintain environmental integrity.

Throughout the sterilization and filling process, the temperature of the composition is maintained within a range of 20 °C to 25 °C, which is critical for preserving the stability of the dispersion and preventing changes in viscosity or phase behavior. Temperature control is achieved through environmental regulation within the cleanroom as well as through temperature-controlled transfer lines and holding vessels. Maintaining this temperature range ensures that the physicochemical properties of the composition remain consistent and that no degradation or structural alteration of the bioactive constituents occurs during handling.

For example, in a representative implementation, 2 liters of final homogenized composition are passed through a 0.22 μm sterile membrane filter at a controlled flow rate under aseptic conditions. The filtered composition is then filled into 100 mL pre-sterilized glass vials within an ISO Class 5 cleanroom, with the ambient temperature maintained at approximately 22 °C. Post-filling analysis confirms absence of microbial contamination and retention of particle size distribution and compositional integrity.

This sterilization approach ensures that the final phytotherapeutic composition meets stringent microbiological quality requirements while maintaining its structural and functional characteristics. The combination of membrane-based sterile filtration and aseptic filling under controlled environmental conditions provides a reliable and scalable method for producing sterile formulations suitable for pharmaceutical or nutraceutical applications.

In an embodiment, the combining, homogenization, and stabilization steps are controlled such that the resulting phytotherapeutic composition comprises total boswellic acids in a concentration range of 25% to 65% by weight of the homogeneous bioactive blend, including 3-acetyl-11-keto-β-boswellic acid present in a concentration between 8% and 30% by weight of the homogeneous bioactive blend, and total guggulsterones present in a concentration between 2% and 12% by weight of the homogeneous bioactive blend, and total guggulsterones present in a concentration between 2% and 12% by weight of the bioactive fraction, wherein the weight ratio of total boswellic acids to total guggulsterones is maintained between 2:1 and 8:1, and wherein the composition further comprises residual solvent content below 0.5% w/w, particle size distribution characterized by a mean diameter below 250 nm with a polydispersity index below 0.35, and a pH maintained between 5.5 and 6.8 through controlled buffering during the stabilization step.

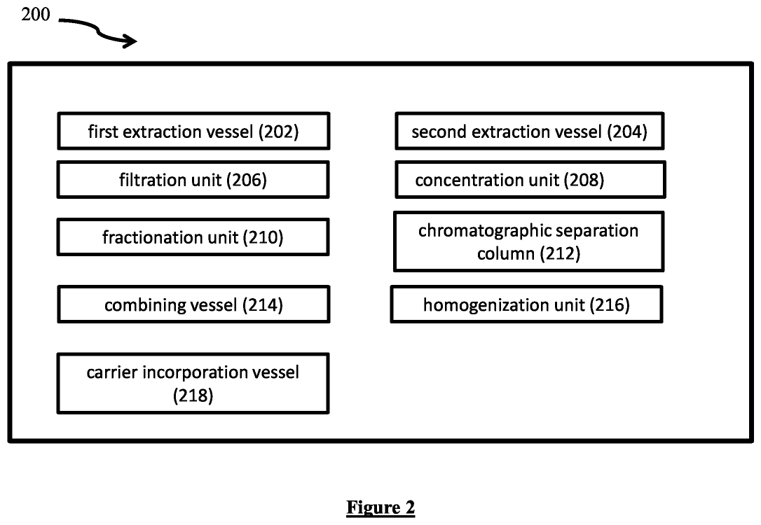

Referring to FIG. 2, a block diagram of a system for synthesizing a phytotherapeutic composition comprising bioactive resin-derived constituents from Boswellia serrata and Commiphora is illustrated. The system 200 comprising: a first extraction vessel (202)comprising an internal agitation assembly and a temperature regulation jacket, the first extraction vessel configured to receive pulverized Boswellia serrata resin and an ethanol-based solvent having a concentration between 70% and 95% v/v, wherein the agitation assembly is configured to operate at variable speeds between 50 and 600 rpm and the temperature regulation jacket is configured to maintain an internal temperature between 45 °C and 65 °C for a duration between 4 and 10 hours to produce a Boswellia extract fraction enriched in boswellic acids; a second extraction vessel (204) comprising an agitation assembly and a temperature control arrangement, the second extraction vessel configured to receive pulverized Commiphora mukul resin and a hydroalcoholic solvent comprising ethanol and water in a volumetric ratio between 60:40 and 90:10, wherein the agitation assembly is configured to operate at speeds between 100 and 600 rpm and the temperature control arrangement is configured to maintain a temperature between 40 °C and 60 °C for a duration between 3 and 8 hours to produce a Commiphora extract fraction enriched in guggulsterones; a filtration unit (206) fluidically coupled to the first extraction vessel and the second extraction vessel, the filtration unit comprising a staged filtration assembly including a coarse filter having pore size between 10 μm and 25 μm and a fine membrane filter having pore size between 0.2 μm and 0.45 μm, configured to remove suspended particulates from the extract fractions; a concentration unit (208) fluidically coupled to the filtration unit, the concentration unit comprising a reduced-pressure chamber configured to operate at a pressure between 80 and 150 mbar and a temperature below 55 °C, the concentration unit including a rotating containment structure configured to rotate between 60 and 120 rpm to remove solvent and generate semi-solid extract masses; a fractionation unit (210)configured to receive the semi-solid Boswellia extract mass, the fractionation unit comprising a solvent handling chamber configured to introduce a non-polar solvent selected from hexane or petroleum ether followed by ethyl acetate under controlled thermal conditions between 60 °C and 75 °C to separate and enrich boswellic acid fractions; a chromatographic separation column (212) configured to receive the Commiphora extract mass, the chromatographic separation column comprising a silica-based stationary phase having particle size between 60 and 120 mesh and a controlled eluent delivery arrangement configured to deliver a solvent gradient of hexane and ethyl acetate in a ratio varying between 9:1 and 7:3 across multiple column volumes to isolate guggulsterone-containing fractions; a combining vessel (214) fluidically coupled to the fractionation unit and the chromatographic separation column, the combining vessel comprising a controlled mixing assembly configured to receive the Boswellia fraction and the Commiphora fraction in a weight ratio between 1:0.5 and 1:2 and to maintain a mixing temperature between 25 °C and 40 °C with agitation between 200 and 500 rpm to produce a homogeneous bioactive blend; a homogenization unit (216) fluidically coupled to the combining vessel, the homogenization unit comprising a high-shear rotor-stator assembly configured to operate at shear rates between 5,000 s⁻¹ and 20,000 s⁻¹ and a high-pressure passage configured to operate between 500 and 1,500 bar across multiple cycles to reduce particle size and produce a stabilized dispersion; and a carrier incorporation vessel (218) configured to introduce a pharmaceutically acceptable carrier comprising at least one of phospholipids, medium-chain triglycerides, or polysorbates in a concentration between 5% and 40% w/w, wherein the carrier incorporation vessel includes a controlled addition mechanism and agitation system configured to maintain uniform dispersion of the bioactive blend within the carrier medium to obtain the phytotherapeutic composition.

The following examples are provided to illustrate specific embodiments of the present invention and to demonstrate the preparation, characterization, and evaluation of phytotherapeutic compositions comprising extracts derived from Boswellia and Commiphora resins. The examples describe detailed experimental procedures including extraction, purification, formulation, and analytical assessment under controlled conditions. It is understood that the described conditions, materials, and parameters are representative and have been selected to enable reproducibility of the disclosed compositions and processes. Variations in process parameters, solvent systems, and operational conditions may be implemented by a person skilled in the art without departing from the scope of the invention as defined by the appended claims.

### Example 1: Preparation of Boswellia Extract Enriched in Boswellic Acids

Dried oleo-gum resin of Boswellia serrata (500 g) was pulverized to obtain particles passing through a 40-mesh sieve. The pulverized material was conditioned at 60% relative humidity and 30 °C for 12 hours. The conditioned resin was transferred to a jacketed extraction vessel, and ethanol (85% v/v, 5 L) was added in two stages, wherein 1.5 L was initially introduced under agitation at 100 rpm for 15 minutes followed by addition of the remaining solvent under agitation at 400 rpm.

Extraction was carried out at 55 °C for 6 hours. The extract was filtered sequentially using a 20 μm filter followed by a 0.45 μm membrane filter. The filtrate was concentrated under reduced pressure at 100 mbar and 50 °C to obtain a semi-solid mass (yield: 312 g).

The semi-solid extract was dispersed in hexane (1:5 w/v) and subjected to ultrasonic treatment at 25 kHz for 30 minutes. After centrifugation at 5,000 rpm for 15 minutes, the insoluble fraction was collected and re-extracted using ethyl acetate under reflux at 70 °C for 2 hours. The resulting fraction was concentrated and subjected to recrystallization using ethyl acetate: methanol (2:1), yielding a boswellic acid-rich fraction.

HPLC analysis indicated: Total boswellic acids: 58.4% w/w; and 3-acetyl-11-keto-β-boswellic acid (AKBA): 21.7% w/w

### Example 2: Preparation of Commiphora Extract Enriched in Guggulsterones

Dried Commiphora mukul resin (400 g) was pulverized and pre-treated with 1% w/v sodium bicarbonate solution (2 L) for 30 minutes, followed by drying at 40 °C.

The treated resin was extracted using ethanol: water (80:20 v/v, 4 L) at 50 °C for 4 hours. The extract was filtered and subjected to a second extraction using ethanol (90% v/v, 3 L) at 55 °C for 3 hours. Combined extracts were cooled to 12 °C at a rate of 2 °C/min to precipitate impurities.

The concentrated extract was partitioned with chloroform (3×1 L). The pooled chloroform fractions were evaporated and purified using silica column chromatography (mesh 100), eluting with hexane: ethyl acetate (8: 2).

Fractions corresponding to Rf 0.45-0.55 were pooled and concentrated.

HPLC analysis indicated: Total guggulsterones (E+Z): 9.6% w/w

### Example 3: Preparation of Combined Phytotherapeutic Composition

The Boswellia fraction (100 g) and Commiphora fraction (40 g) were separately dissolved in ethanol: propylene glycol (2:1 v/v). The Commiphora solution was added dropwise into the Boswellia solution at 3 mL/min under stirring at 350 rpm and temperature maintained at 35 °C

A carrier phase comprising medium-chain triglycerides (20% w/w) and phosphatidylcholine (5% w/w) was prepared at 50 °C The bioactive mixture was incorporated into the carrier phase under stirring at 500 rpm.

The mixture was subjected to high-shear homogenization at 10,000 rpm for 10 minutes followed by high-pressure homogenization at 1,000 bar for 5 cycles.

The resulting nano-dispersion exhibited:
1) Mean particle size: 182 nm
2) Polydispersity index: 0.28
3) pH: 6.2

### Example 4: in Vitro Anti-inflammatory Activity (cytokine Inhibition Assay)

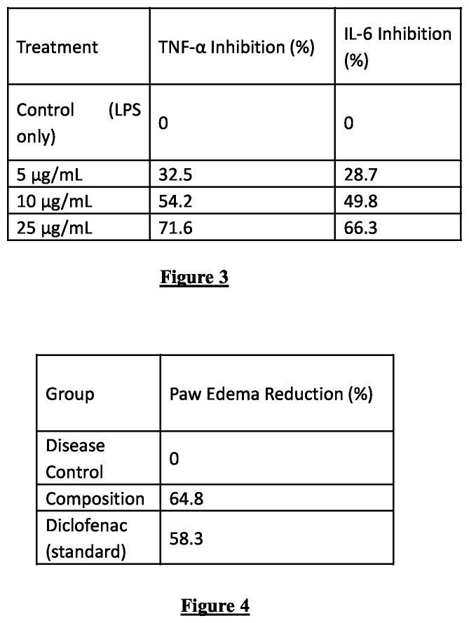

Murine macrophage cells (RAW 264.7) were stimulated using lipopolysaccharide (LPS) at a concentration of 1 μg/mL to induce inflammatory cytokine production. The cells were subsequently treated with the synthesized phytotherapeutic composition at concentrations of 5 μg/mL, 10 μg/mL, and 25 μg/mL. Levels of pro-inflammatory cytokines including tumor necrosis factor-alpha (TNF-α) and interleukin-6 (IL-6) were quantified using enzyme-linked immunosorbent assay (ELISA). The measured cytokine inhibition values obtained for different treatment concentrations are presented in FIG. 3.

In a comparative evaluation, a reference anti-inflammatory compound, namely Diclofenac administered at a concentration of 10 μg/mL, demonstrated TNF-α inhibition of 52.1% and IL-6 inhibition of 48.5%. The synthesized phytotherapeutic composition exhibited concentration-dependent cytokine suppression, wherein treatment at 10μg/mL produced inhibition values exceeding those observed for the reference compound, thereby indicating enhanced anti-inflammatory efficacy.

### Example 5: IN Vivo Anti-Arthritic Activity

Wistar rats (n=6 per group) were subjected to Freund's complete adjuvant-induced arthritis for evaluation of anti-arthritic efficacy. The synthesized phytotherapeutic composition was orally administered at a dosage of 100 mg/kg body weight for a treatment duration of 21 days. Reduction in paw edema was measured periodically and the comparative anti-inflammatory response observed for the treated groups is presented in FIG. 4.

A disease control group exhibited no measurable reduction in paw edema, whereas animals treated with the synthesized phytotherapeutic composition demonstrated a paw edema reduction of 64.8%. A comparative standard treatment group administered Diclofenac exhibited a paw edema reduction of 58.3% under corresponding experimental conditions.

Histopathological examination of synovial tissue and cartilage structures further demonstrated reduced inflammatory cell infiltration, decreased synovial hyperplasia, and lower cartilage degradation in animals treated with the synthesized phytotherapeutic composition compared to the untreated disease control group, thereby indicating significant anti-arthritic and tissue-protective activity.

### Example 6: Oxidative Stress Reduction Assay

The antioxidant potential of the synthesized phytotherapeutic composition was evaluated using a 2,2-diphenyl-1-picrylhydrazyl (DPPH) radical scavenging assay. Different concentrations of the composition were incubated with the DPPH radical solution under controlled conditions, and the reduction in absorbance corresponding to free radical neutralization was measured spectrophotometrically.

The synthesized phytotherapeutic composition demonstrated an IC50 value of 18.2 μg/mL, indicating substantial free radical scavenging activity. Under corresponding assay conditions, Ascorbic acid used as a reference antioxidant standard exhibited an IC50 value of 15.6 μg/mL. The observed antioxidant activity of the synthesized composition demonstrates effective oxidative stress reduction capability attributable to the synergistic action of boswellic acids and guggulsterones present within the formulation.

### Example 7: Stability Study

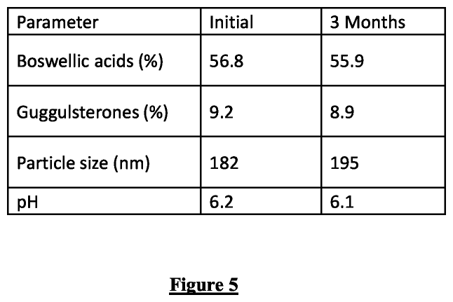

The synthesized phytotherapeutic composition was subjected to accelerated stability evaluation under controlled environmental conditions maintained at 40 °C±2 °C and 75% relative humidity ±5% for a storage duration of 3 months. Periodic analysis was performed to evaluate physicochemical stability, retention of active constituents, particle size distribution, and pH stability. The comparative analytical values obtained at the initial time point and after 3 months of storage are presented in FIG. 5.

The composition exhibited minimal variation in total boswellic acid concentration, decreasing from 56.8% to 55.9%, while total guggulsterone concentration decreased from 9.2% to 8.9% over the study period. Mean particle size showed only a minor increase from 182 nm to 195 nm, indicating maintenance of nanoscale dispersion stability. Similarly, pH remained substantially stable, changing from 6.2 to 6.1 during storage.

Visual and microscopic examination of the composition following accelerated storage conditions revealed no observable phase separation, sedimentation, or precipitation, thereby demonstrating substantial physicochemical stability and retention of formulation integrity under accelerated environmental stress conditions.

The following comparative examples are provided to evaluate the performance of compositions prepared using conventional extraction and formulation techniques in comparison with the compositions obtained through the process described herein. The comparative examples are carried out under conditions that do not incorporate one or more of the controlled parameters, sequential processing steps, or purification procedures defined in the present invention. The results obtained from these comparative examples are analyzed using identical analytical and biological evaluation methods to enable direct comparison.

### Comparative Example 1: Direct Ethanol Extraction Without Pre-Conditioning

Dried oleo-gum resin of Boswellia serrata (500 g) was directly subjected to extraction using ethanol (90% v/v, 5 L) at 60 °C for 6 hours under agitation at 300 rpm without prior humidity conditioning or staged solvent introduction. The resulting extract, upon concentration under reduced pressure, yielded a semi-solid mass exhibiting incomplete solubilization of resin fractions. High-performance liquid chromatography analysis showed total boswellic acid content of 41.2% w/w, with 3-acetyl-11-keto-β-boswellic acid (AKBA) limited to 11.3% w/w. In contrast, the process employing controlled pre-conditioning and staged solvent addition yielded 58.4% total boswellic acids and 21.7% AKBA, indicating a relative increase of approximately 41.7% in total boswellic acids and 92% in AKBA concentration. The reduced yield and lower enrichment in the absence of controlled pre-conditioning indicate inefficient diffusion and incomplete extraction of active constituents.

### Comparative Example 2: Single-stage Commiphora Extraction

Pulverized Commiphora mukul resin (400 g) was extracted using a single-stage hydroalcoholic solvent system (ethanol: water, 70:30 v/v) at 50 °C for 6 hours, followed by direct concentration. The resulting extract showed total guggulsterone content of 5.1% w/w as determined by HPLC analysis. In comparison, the sequential dual-stage extraction combined with controlled cooling precipitation yielded 9.6% w/w guggulsterones. The single-stage process therefore resulted in a reduction of approximately 46.9% in active constituent concentration, demonstrating that absence of staged extraction and impurity removal significantly limits enrichment efficiency.

### Comparative Example 3: Absence of Selective Fractionation

Boswellia extract obtained via ethanol extraction was concentrated and used without hexane defatting or ethyl acetate re-extraction. The resulting composition contained 38.6% w/w total boswellic acids and 9.8% w/w AKBA. When compared with the fractionated extract containing 58.4% total boswellic acids and 21.7% AKBA, the non-fractionated process resulted in reductions of approximately 33.9% and 54.8% respectively. The data establishes that selective removal of non-polar terpenoids and targeted enrichment steps significantly influence the final composition.

### Comparative Example 4: Absence of Chromatographic Purification

The Commiphora extract obtained after solvent partitioning was used directly without silica-based chromatographic separation. HPLC analysis indicated total guggulsterone content of 4.3% w/w, along with additional unresolved peaks representing co-extracted impurities. In contrast, chromatographically purified fractions exhibited 9.6% w/w guggulsterones with a defined peak profile. The absence of chromatographic separation therefore resulted in a reduction of approximately 55.2% in target compound concentration and reduced compositional specificity.

### Comparative Example 5: Uncontrolled Blending of Extracts

Boswellia and Commiphora extracts were directly blended in a 1:1 ratio at ambient temperature without co-solvent-assisted dissolution or controlled addition rate. Content uniformity analysis conducted across multiple samples indicated variation in active constituent distribution of ±18%, whereas the controlled blending process exhibited variation within ±3%. This represents a six-fold increase in compositional inconsistency, demonstrating that uncontrolled mixing results in non-uniform distribution of bioactive compounds.

### Comparative Example 6: Absence of High-shear and High-pressure Homogenization

The combined extract was mixed with carrier oil using conventional mechanical stirring at 500 rpm for 20 minutes without application of high-shear or high-pressure homogenization. Particle size analysis revealed a mean particle diameter of 2.8 μm with a polydispersity index of 0.72. In contrast, the multi-stage homogenization process produced a mean particle size of 182 nm with a polydispersity index of 0.28. This corresponds to a reduction in particle size of over 93% and a significant narrowing of particle distribution, indicating that conventional mixing fails to achieve nanoscale dispersion.

### Comparative Example 7: Absence of Carrier-assisted Formulation

The purified extracts were evaluated without incorporation into a carrier system. Dissolution testing in simulated intestinal fluid showed a dissolved fraction of 22.4% after 2 hours. The carrier-assisted composition demonstrated a dissolved fraction exceeding 68.5% under identical conditions. The absence of carrier incorporation therefore resulted in a reduction of approximately 67% in solubilized fraction, indicating poor bioavailability of the conventional preparation.

### Comparative Example 8: in Vitro Anti-inflammatory Activity

The conventionally prepared extract was tested in LPS-stimulated RAW 264.7 macrophage cells at a concentration of 10 μg/mL. The extract produced TNF-α inhibition of 34.7% and IL-6 inhibition of 31.2%. Under identical conditions, the composition produced using the defined process exhibited TNF-α inhibition of 54.2% and IL-6 inhibition of 49.8%. This represents increases of approximately 56.2% and 59.6% respectively, indicating enhanced biological activity linked to compositional refinement.

### Comparative Example 9: in Vivo Anti-arthritic Activity

In a Freund's complete adjuvant-induced arthritis model in rats, administration of the conventional extract at 100 mg/kg resulted in a paw edema reduction of 42.6% after 21 days. The composition prepared using the defined process demonstrated 64.8% reduction under identical conditions. This corresponds to an improvement of approximately 52.1% in therapeutic efficacy, correlating with enhanced concentration and bioavailability of active constituents.

### Comparative Example 10: Stability Assessment

The conventionally prepared formulation was subjected to accelerated stability testing at 40 °C and 75% relative humidity for 3 months. The boswellic acid content decreased from 40.2% to 34.5%, and guggulsterone content decreased from 5.0% to 3.8%, representing reductions of 14.2% and 24% respectively. In contrast, the stabilized composition prepared using the defined process showed reductions below 2% for both constituents under identical conditions. The conventional formulation additionally exhibited phase separation, indicating instability in the absence of controlled formulation steps.

In an embodiment, the combining, homogenization, and stabilization stages applied to the bioactive fractions derived from Boswellia serrata and Commiphora mukul are executed under tightly regulated compositional and process conditions such that the final phytotherapeutic composition exhibits a defined quantitative profile of active constituents together with controlled physicochemical properties. The Boswellia fraction, enriched in boswellic acids, and the Commiphora fraction, enriched in guggulsterones, are first quantitatively analyzed using chromatographic techniques such as high-performance liquid chromatography to determine their respective concentrations prior to blending. Based on these measurements, the fractions are proportioned during the combining stage in a manner that ensures the total boswellic acid content in the resulting bioactive fraction lies within the range of 25% to 65% by weight, while maintaining the concentration of 3-acetyl-11-keto-β-boswellic acid within a narrower range of 8% to 30% by weight. Simultaneously, the Commiphora-derived fraction is incorporated in a quantity sufficient to yield total guggulsterones in the range of 2% to 12% by weight, with the overall ratio of boswellic acids to guggulsterones controlled between 2:1 and 8:1 through precise adjustment of input masses and monitored blending.

Following controlled combining, the mixture is subjected to homogenization under defined shear and pressure conditions to ensure that the active constituents are uniformly dispersed within the carrier medium at a nanoscale level. The homogenization parameters, including rotor-stator speed, high-pressure cycles, and optional microfluidization, are adjusted based on real-time particle size measurements to achieve a mean particle diameter below 250 nm with a polydispersity index below 0.35. This narrow particle size distribution reflects a uniform dispersion in which the active compounds are evenly distributed throughout the carrier system, thereby minimizing the likelihood of aggregation or phase separation. The consistency of this dispersion is verified through dynamic light scattering analysis and maintained through controlled processing conditions, including temperature regulation and staged energy input.

During the stabilization phase, the composition is further conditioned to ensure long-term physical and chemical consistency. Residual solvent content is reduced to below 0.5% w/w through controlled evaporation and vacuum drying steps, with verification performed using analytical techniques such as gas chromatography. The pH of the composition is adjusted and maintained within a range of 5.5 to 6.8 using a buffering system, typically comprising citric acid and sodium citrate, which is introduced under controlled conditions to achieve uniform distribution without disrupting the dispersion structure. The selected pH range ensures compatibility with biological systems while maintaining stability of the active constituents and preventing hydrolytic degradation.

For example, in a representative implementation, a Boswellia fraction containing 55% total boswellic acids and a Commiphora fraction containing 8% guggulsterones are combined in a ratio of 3:1 by weight, resulting in a final composition containing approximately 45% boswellic acids and 6% guggulsterones. The mixture is homogenized to achieve a mean particle size of 180 nm with a polydispersity index of 0.28, and subsequent solvent removal reduces residual ethanol content to 0.3% w/w. The pH is adjusted to 6.2 using a citrate buffer system, yielding a stable dispersion with no observable phase separation over extended storage.

The coordinated control of compositional ratios, particle size distribution, residual solvent levels, and pH ensures that the resulting phytotherapeutic composition maintains uniformity, stability, and reproducibility across batches. The interplay between these parameters enables the formation of a well-defined formulation in which the bioactive constituents are consistently distributed and maintained within specified limits, thereby supporting reliable downstream application and performance.

The present invention is implemented through a coordinated sequence of mechanically controlled operations governed by a process control technique configured to regulate extraction, purification, blending, and stabilization of bioactive constituents derived from Boswellia serrata and Commiphora mukul. The technique is executed by a programmable processing unit operatively coupled to sensing elements and actuating components distributed across the system, wherein the processing unit continuously receives input signals corresponding to temperature, pressure, agitation speed, solvent flow rate, viscosity, and concentration parameters, and dynamically adjusts operational conditions to maintain predefined process thresholds.

The technique initiates with a raw material conditioning routine in which input parameters including resin particle size, moisture content, and bulk density are detected using integrated sensing assemblies. Based on these inputs, the technique computes optimal pre-conditioning conditions, including humidity exposure duration and temperature setpoints, to induce controlled swelling of the resin matrix and enhance solvent penetration. The processing unit then regulates the staged introduction of solvent into the extraction vessels, wherein an initial wetting phase is executed by introducing a fraction of the total solvent volume at a lower agitation speed to prevent agglomeration, followed by a progressive increase in solvent flow and agitation intensity to facilitate diffusion-driven mass transfer of boswellic acids and guggulsterones into the solvent medium.

During the extraction phase, the technique employs a feedback-controlled thermal regulation loop in which real-time temperature measurements are compared against predefined setpoints, and heating or cooling inputs to the vessel jacket are modulated accordingly to maintain a stable thermal environment. Simultaneously, agitation speed is dynamically adjusted based on viscosity feedback and diffusion rate estimations, thereby ensuring uniform solvent-resin interaction without inducing excessive shear that could degrade sensitive constituents. The technique further incorporates time-dependent extraction profiling, wherein solvent contact duration is optimized based on concentration gradients detected through inline spectroscopic or conductivity measurements.

Following extraction, the technique transitions to a filtration control routine in which pressure differentials across staged filtration assemblies are monitored to detect clogging or flow resistance. Based on these measurements, the processing unit adjusts pump speeds and flow rates to maintain consistent filtration throughput while preventing damage to filter media. The filtered extracts are then directed to a concentration unit, where the technique regulates reduced-pressure conditions and rotational dynamics to achieve controlled solvent evaporation. The technique executes solvent removal in cyclic intervals, incorporating cooling phases between evaporation cycles to prevent localized overheating and thermal degradation. Residual solvent levels are continuously estimated through condensate volume tracking and vapor composition sensing, enabling precise termination of the concentration process upon reaching target thresholds.

In the fractionation stage, the technique controls the introduction of non-polar and polar solvents in a sequential manner, while simultaneously regulating ultrasonic energy input and thermal conditions to facilitate selective dissolution and separation of target compounds. Phase separation is monitored through density and turbidity sensors, and centrifugation parameters are adjusted to achieve efficient segregation of fractions. For chromatographic purification, the technique governs the delivery of solvent gradients through a programmable eluent control system, wherein flow rates and solvent ratios are dynamically varied based on real-time detection of compound elution profiles using optical or UV-based sensing. Fraction collection is synchronized with retention factor ranges calculated by the processing unit, ensuring selective isolation of guggulsterone-enriched fractions.

The technique further manages the combining phase by calculating optimal blending ratios of Boswellia and Commiphora fractions based on concentration data obtained from upstream processes. Controlled addition rates are maintained through precision flow regulators, and mixing conditions including temperature and agitation speed are adjusted to ensure molecular-level dispersion of bioactive constituents without precipitation. The system then enters the homogenization phase, where the technique coordinates a multi-stage shear process involving both rotor-stator mixing and high-pressure homogenization. Shear rates, pressure levels, and cycle counts are dynamically adjusted based on real-time particle size measurements obtained through inline particle size analyzers, ensuring achievement of a target nanoscale distribution with minimal polydispersity.

During carrier incorporation, the technique regulates the sequential addition of lipid, surfactant, and aqueous phases, ensuring controlled formation of vesicular or emulsified structures encapsulating the bioactive compounds. Hydration rates, stirring speeds, and temperature conditions are continuously optimized to promote stable dispersion and prevent phase separation. The technique also incorporates a thermal management subroutine, wherein cooling cycles are introduced during high-energy processing stages to prevent degradation of thermolabile constituents.

Subsequent stabilization steps are governed by a chemical control routine in which pH levels are continuously monitored and adjusted through controlled addition of buffering agents. Antioxidant incorporation is executed based on oxidative stability predictions derived from oxygen exposure data and processing duration. The technique further manages deaeration processes by controlling vacuum levels and exposure time to remove entrapped gases and enhance compositional stability.

For optional downstream processes such as spray drying or microencapsulation, the technique regulates parameters including inlet and outlet temperatures, feed rates, atomization pressure, and crosslinking conditions to produce stable particulate forms with controlled moisture content and release characteristics. Throughout the entire process, the technique maintains a closed-loop control architecture, continuously comparing real-time process data with predefined models and adjusting operational parameters to ensure consistency, efficiency, and product quality.

The integration of this technique with the mechanical system enables seamless coordination of multiple processing stages, eliminates manual intervention, and ensures reproducibility across production cycles. By incorporating adaptive control strategies and real-time monitoring, the invention achieves a high degree of precision in the extraction and formulation of phytotherapeutic compositions, thereby overcoming the limitations of conventional methods and enabling scalable, industrial-grade manufacturing.

The present invention discloses a highly controlled phytotherapeutic synthesis process in which dried oleo-gum resin of Boswellia serrata is subjected to size reduction followed by solvent extraction using an ethanol-based solvent system under regulated thermal and agitation conditions. The extraction is performed in a mechanically agitated vessel equipped with a temperature-regulated jacket, wherein solvent penetration and diffusion of boswellic acids are optimized through staged solvent addition and controlled agitation speeds. The extracted solution is subsequently subjected to multi-stage filtration and reduced-pressure concentration to obtain a semi-solid extract mass while minimizing thermal degradation and oxidative exposure.

In parallel, Commiphora mukul resin is processed through a dual-phase extraction protocol using hydroalcoholic solvents under controlled temperature conditions to selectively solubilize guggulsterones and associated bioactive compounds. The extract is cooled under controlled conditions to induce precipitation of impurities, followed by filtration and chromatographic purification using a silica-based stationary phase and gradient solvent elution to isolate fractions enriched in E-and Z-guggulsterones.

The Boswellia extract undergoes further fractionation involving selective removal of non-polar terpenoids using hexane or petroleum ether, followed by ethyl acetate extraction to concentrate specific boswellic acids such as 3-acetyl-11-keto-β-boswellic acid. The purified Boswellia and Commiphora fractions are subsequently combined under controlled temperature and mixing conditions to form a homogeneous bioactive blend.

The combined blend is incorporated into a carrier medium comprising phospholipids, triglycerides, or surfactants, followed by high-shear and high-pressure homogenization to produce a nano-scale dispersion with controlled particle size distribution. The process further includes optional steps such as microencapsulation, spray drying, solvent removal, and stabilization through pH adjustment and antioxidant addition, resulting in a pharmaceutically stable composition.

The invention further discloses a structurally integrated system configured as a multi-unit processing apparatus for synthesizing the phytotherapeutic composition. The system comprises a first extraction vessel constructed as a sealed cylindrical chamber fabricated from corrosion-resistant material and equipped with an internal impeller-driven agitation assembly and an external thermal jacket configured for circulating a heating medium. The vessel includes controlled solvent inlet ports, humidity conditioning interfaces, and programmable speed regulation mechanisms for staged agitation.

A second extraction vessel is provided for processing Commiphora mukul resin, wherein the vessel includes independent thermal control circuits and variable-speed agitation assemblies configured to maintain precise extraction conditions for hydroalcoholic solvents. Both extraction vessels are fluidically connected through sealed conduits to a filtration unit comprising a dual-stage filtration assembly with replaceable filter media arranged in series to enable sequential removal of coarse and fine particulates.

The filtered extract streams are directed into a concentration unit comprising a reduced-pressure chamber integrated with a rotating containment structure, wherein solvent evaporation is performed under controlled pressure and rotational speed conditions. The system includes a vacuum generation assembly and condensate recovery channels for efficient solvent removal and recycling.

A fractionation unit is operatively coupled to the concentration unit and comprises a solvent handling chamber equipped with ultrasonic transducers and temperature-controlled reflux systems configured to selectively separate non-polar and polar fractions. A chromatographic separation column is integrated within the system, comprising a vertically oriented cylindrical column packed with silica material and connected to a programmable eluent delivery system capable of generating solvent gradients with precise flow control.

A combining vessel is provided downstream of the fractionation and chromatographic units, wherein the vessel includes a controlled mixing assembly with variable-speed impellers and temperature sensors configured to maintain uniform blending conditions. The combining vessel is connected to a homogenization unit comprising a rotor-stator assembly and a high-pressure homogenization passage, wherein the system is configured to achieve particle size reduction through controlled shear and pressure cycling.

A carrier incorporation vessel is integrated with the homogenization unit and includes heating elements, surfactant dissolution chambers, and controlled addition mechanisms for introducing carrier media. The system further includes microfluidization channels, cooling jackets, and inline particle size monitoring sensors configured to ensure uniform dispersion characteristics.

The entire system is structurally supported on a modular frame comprising vibration-dampening mounts and interconnected piping networks designed for aseptic operation. Control of the system is achieved through a programmable processing unit configured to regulate temperature, pressure, flow rate, agitation speed, and processing time across all units, thereby ensuring reproducibility and process consistency.

The system described herein is implemented through a physically integrated assembly of processing equipment configured for sequential handling of resin-derived materials, wherein each functional element corresponds to a structurally defined unit capable of performing a specific physicochemical operation. The first extraction vessel and the second extraction vessel are constructed as closed reaction chambers equipped with mechanically driven agitation assemblies, such as shaft-mounted impellers coupled to variable-speed drive mechanisms, and jacketed walls adapted for circulation of heating or cooling media to maintain defined temperature ranges during solvent extraction. The filtration unit comprises a pressure-driven filtration housing containing replaceable filter elements arranged in series, including coarse filtration media and fine membrane barriers, enabling removal of suspended particulates through controlled fluid flow paths. The concentration unit is formed as a reduced-pressure evaporation chamber incorporating a sealed enclosure, a vacuum-generating device for maintaining sub-atmospheric pressure, and a rotatable containment structure mounted on a motor-driven axis to promote uniform solvent removal under controlled thermal conditions. The fractionation unit includes a solvent-resistant chamber with inlet and outlet conduits, heating elements, and controlled solvent introduction ports to facilitate sequential contact with immiscible solvents under regulated temperature conditions for selective separation of resin constituents. The chromatographic separation column is configured as a vertically oriented column packed with a defined particulate stationary phase, coupled with a solvent delivery arrangement comprising flow-regulated conduits and gradient-forming reservoirs that enable controlled passage of eluents through the packed bed. The combining vessel is a mechanically agitated mixing chamber equipped with temperature-regulated walls and inlet ports for receiving multiple streams, allowing controlled blending under defined agitation speeds. The homogenization unit comprises a rotor-stator assembly driven by a high-speed motor to generate defined shear rates, in combination with a high-pressure passage formed by a narrow flow channel connected to a pressure-generating device, enabling repeated passage of the mixture under elevated pressure conditions to achieve particle size reduction. The carrier incorporation vessel includes a dosing arrangement with metered inlet lines and an agitation system to ensure uniform distribution of carrier components within the processed mixture.

The disclosed process and system are suitable for large-scale industrial production of phytotherapeutic formulations with consistent quality and enhanced bioavailability. The integration of mechanical processing units with controlled environmental parameters enables efficient extraction, purification, and stabilization of plant-derived bioactive compounds. The system is particularly applicable in pharmaceutical manufacturing, nutraceutical production, and herbal formulation industries requiring standardized compositions with reproducible therapeutic efficacy.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A process for synthesizing a phytotherapeutic composition comprising bioactive resin-derived constituents from Boswellia and Commiphora, the process comprising:
subjecting dried oleo-gum resin of Boswellia serrata to size reduction followed by solvent extraction using an organic solvent system comprising ethanol in a concentration range of 70-95% v/v, maintaining a solid-to-solvent ratio between 1:5 and 1:15 (w/v), and performing extraction under continuous agitation at a temperature maintained between 45 °C and 65 °C for a duration of 4 to 10 hours to obtain a Boswellia extract fraction enriched in boswellic acids;
subjecting dried oleo-gum resin of Commiphora mukul to pulverization followed by extraction using a hydroalcoholic solvent comprising ethanol and water in a volumetric ratio between 60:40 and 90:10, maintaining a solid-to-solvent ratio between 1:4 and 1:12 (w/v), and conducting extraction at a temperature between 40 °C and 60 °C for a duration of 3 to 8 hours to obtain a Commiphora extract fraction enriched in guggulsterones;
filtering each extract fraction through a membrane filter having a pore size between 0.2 μm and 5 μm followed by concentration under reduced pressure at a temperature below 55 °C to obtain a semi-solid Boswellia extract mass and a semi-solid Commiphora extract mass;
subjecting the semi-solid Boswellia extract mass to a fractionation step using a non-polar solvent selected from hexane or petroleum ether to remove volatile terpenoids, followed by re-extraction with ethyl acetate to obtain a purified Boswellia fraction containing 3-acetyl-11-keto-β-boswellic acid in a concentration range of 10-40% by weight of the fraction;
subjecting the semi-solid Commiphora extract mass to chromatographic purification using a silica-based stationary phase and an eluent system comprising hexane and ethyl acetate in a ratio between 7:3 and 9:1 to o obtain a purified Commiphora fraction containing E-and Z-guggulsterones in a combined concentration range of 2-15% by weight of the fraction;
combining the purified Boswellia fraction and the purified Commiphora fraction in a weight ratio ranging from 1:0.5 to 1:2 under controlled mixing conditions at a temperature between 25 °C and 40 °C to form a homogeneous bioactive blend;
incorporating the homogeneous bioactive blend into a carrier medium comprising at least one pharmaceutically acceptable excipient selected from phospholipids, medium-chain triglycerides, or polysorbates in a concentration range of 5-40% w/w, followed by a high-shear homogenization at a shear rate between 5,000 s⁻¹ and 20,000 s⁻¹ for a duration of 10 to 30 minutes to obtain a stabilized phytotherapeutic composition, wherein the extraction of the resin of the Boswellia serrata further comprises pre-conditioning the size-reduced resin by exposing it to a controlled humidity environment maintained at 55-70% relative humidity and a temperature between 28 °C and 35 °C for a duration of 6 to

18. hours prior to solvent contact, followed by gradual addition of the ethanol in a staged manner comprising an initial wetting phase using 20-30% of total solvent volume introduced over 10-20 minutes under low-speed agitation between 50 and 150 rpm, and a subsequent extraction phase in which the remaining solvent volume is introduced continuously while increasing agitation speed to 300-600 rpm to facilitate controlled diffusion of boswellic acids into the solvent, wherein the extraction of the resin of Commiphora mukul includes a sequential dual-phase extraction protocol comprising an initial extraction stage using ethanol-water mixture at a ratio of 70:30 conducted at 40-45 °C for 2 to 4 hours to solubilize polar constituents, followed by separation of solvent phase and re-extraction of residual solid mass using ethanol concentration of 85-95% v/v at 50-60 °C for an additional 2 to 5 hours, wherein combined extract fractions are subjected to controlled cooling at a rate of 1-3 °C per minute to a final temperature of 10-15 °C to induce precipitation of high molecular weight resinous impurities prior to filtration, and wherein the concentration under reduced pressure is performed using a rotary evaporation system operating at a pressure between 80 and 150 mbar and a rotation speed between 60 and 120 rpm, wherein the solvent removal is conducted in incremental cycles each lasting 10-20 minutes with intermittent cooling phases of 5-10 minutes to maintain an internal temperature of the extract mass below 50 °C , followed by nitrogen purging at a flow rate between 0.5 and 2 L/min for 10 to 30 minutes to remove residual solvent traces and minimize oxidative degradation of bioactive constituents.

2. The process of claim 1, wherein the fractionation of the Boswellia extract mass includes dispersing concentrated extract in hexane at a ratio of 1:3 to 1:8 (w/v) and subjecting the dispersion to ultrasonic agitation at a frequency between 20 kHz and 40 kHz and power density between 100 and 300 W/L for 15 to 45 minutes to facilitate selective dissolution of non-polar terpenoids, followed by phase separation through centrifugation at 3,000 to 8,000 rpm for 10 to 25 minutes, and subsequent recovery of an insoluble fraction which is then re-extracted using ethyl acetate under reflux conditions at 60-75 °C for 1 to 3 hours to enrich boswellic acid concentration.

3. The process of claim 1, wherein the chromatographic purification of the Commiphora extract includes packing a silica column having particle size between 60 and 120 mesh and conditioning the column using hexane prior to sample loading, followed by gradient elution initiated with hexane: ethyl acetate ratio of 9:1 and gradually shifted to 7:3 over a volume range of 5 to 15 column volumes, wherein fractions are collected at intervals of 10 to 25 mL and monitored using thin-layer chromatography with a mobile phase comprising hexane: ethyl acetate (8:2) and visualized under UV light at 254 nm, followed by pooling of fractions exhibiting retention factor values between 0.3 and 0.6 corresponding to guggulsterones.

4. The process of claim 1, wherein the combining of the Boswellia fraction and the Commiphora fraction is performed by initially dissolving each fraction separately in a co-solvent system comprising ethanol and propylene glycol in a volumetric ratio between 1:1 and 3:1, followed by gradual addition of a Commiphora solution into a Boswellia solution at a controlled rate of 1 to 5 mL per minute under continuous stirring at 200 to 500 rpm to obtain a mixture, while maintaining a temperature of the mixture between 30 °C and 40 °C to prevent localized precipitation and ensure molecular-level dispersion of active constituents.

5. The process of claim 1, wherein the high-shear homogenization includes subjecting combined bioactive blend and carrier medium to a two-stage shear process comprising a primary homogenization at 8,000-12,000 rpm for 5 -15 minutes followed by high-pressure homogenization at a pressure between 500 and 1,500 bar for 3 to 10 cycles, wherein each cycle includes passage through a homogenization valve with a gap size between 50 and 200 micrometers to produce a uniform dispersion with droplet size below 200 nm.

6. The process of claim 1, wherein the carrier medium further includes incorporation of phosphatidylcholine in a concentration between 2% and 10% w/w, wherein the phosphatidylcholine is pre-dissolved in ethanol and added to the bioactive blend prior to homogenization, followed by controlled hydration using purified water added dropwise at a rate of 0.5 to 2 mL per minute under stirring at 300 to 700 rpm to facilitate formation of lipid-based vesicular structures encapsulating active constituents.

7. The process of claim 1, wherein the stabilized phytotherapeutic composition is subjected to a post-processing stabilization step comprising adjusting pH to a range between 5.5 and 6.8 using a buffering system comprising citric acid and sodium citrate, followed by addition of an antioxidant selected from tocopherol or ascorbyl palmitate in a concentration between 0.01% and 0.5% w/w, and subsequently performing deaeration under vacuum at 50 to 100 mbar for 10 to 20 minutes to remove entrapped air and enhance compositional stability.

8. The process of claim 1, wherein the process further includes a controlled drying step applied to the stabilized phytotherapeutic composition by spray drying using an inlet temperature between 120 °C and 160 °C and an outlet temperature between 60 °C and 90 °C, wherein a feed rate is maintained between 3 and 10 mL/min and atomization is performed using a nozzle pressure between 1.5 and 3 bar to obtain a free-flowing powder with moisture content below 5% w/w.

9. The process of claim 1, wherein the process includes a microencapsulation step comprising dispersing the stabilized phytotherapeutic composition in an aqueous solution containing a polymer selected from poly(lactic-co-glycolic acid) or sodium alginate at a concentration between 1% and 5% w/v, followed by emulsification using a surfactant at 0.5% to 2% w/v and subsequent crosslinking using calcium chloride solution at 0.1 to 0.5 M concentration under continuous stirring at 300 to 800 rpm for 20 to 60 minutes to form encapsulated particles with controlled release characteristics.

10. The process of claim 1, wherein the Boswellia extract fraction obtained after ethyl acetate re-extraction is subjected to a controlled recrystallization step comprising dissolving the fraction in a mixed solvent system of ethyl acetate and methanol in a volumetric ratio between 3:1 and 1:1 at a temperature between 55 °C and 70 °C to form a clear solution, followed by gradual cooling at a controlled rate of 0.5 to 2 °C per minute to a temperature between 5 °C and 15 °C while maintaining continuous agitation at 100 to 300 rpm, and isolating crystalline precipitates enriched in 3-acetyl-11-keto-β-boswellic acid through vacuum filtration and drying under reduced pressure at 30-45 °C for 6 to 12 hours.

11. The process of claim 1, wherein the Commiphora extract fraction, prior to chromatographic purification, is subjected to liquid-liquid partitioning by dispersing the concentrated extract in distilled water at a ratio of 1:2 to 1:6 (w/v), followed by sequential extraction using n-hexane and chloroform, each applied in two to four cycles with solvent-to-aqueous phase ratios between 1:1 and 3:1, wherein each extraction cycle includes agitation at 200 to 600 rpm for 15 to 30 minutes and phase separation by settling for 20 to 40 minutes, and pooling chloroform fractions for subsequent purification of guggulsterones.

12. The process of claim 1, wherein the filtration of the Boswellia and Commiphora extract fractions further includes a two-stage clarification procedure comprising an initial coarse filtration using a filter medium having pore size between 10 μm and 25 μm followed by fine filtration using a membrane filter having pore size between 0.2 μm and 0.45 μm, wherein the filtration is conducted under a pressure differential between 0.5 and 2 bar and maintained at a temperature between 25 °C and 35 °C to limit viscosity-induced flow resistance.

13. The process of claim 1, wherein the concentration under reduced pressure further includes real-time monitoring of solvent removal by measuring condensate volume and correlating it with initial solvent input, and terminating the concentration upon achieving a residual solvent content below 5% w/w, followed by immediate transfer of the semi-solid extract into an inert atmosphere chamber maintained with nitrogen or argon at oxygen levels below 2% to limit oxidative transformation of active constituents.

14. The process of claim 1, wherein the combining of the Boswellia and Commiphora fractions further includes a pre-mixing equilibration step comprising holding individual fractions at a temperature between 35 °C and 45 °C for 20 to 60 minutes to equalize viscosity, followed by introduction of a co-solubilizing agent selected from polyethylene glycol having molecular weight between 200 and 600 in a concentration between 5% and 20% w/w prior to blending, and performing mixing under a controlled shear rate between 100 s⁻¹ and 500 s⁻¹ to obtain a uniform intermediate mixture.

15. The process of claim 1, wherein the homogenization step further includes temperature regulation by circulating a cooling medium through a jacketed vessel to maintain a composition temperature within a range of 20 °C to 30 °C during high-shear processing, and intermittently pausing homogenization cycles for 2 to 5 minutes after every 5 to 10 minutes of operation to prevent thermal degradation of thermolabile constituents.

16. The process of claim 1, wherein the carrier medium preparation includes pre-dissolving surfactants selected from polysorbate 80 or lecithin in a lipid phase comprising medium-chain triglycerides at a temperature between 40 °C and 60 °C , followed by gradual addition of an aqueous phase maintained at 25 °C to 35 °C under continuous stirring at 400 to 900 rpm to form a pre-emulsion prior to incorporation of the bioactive blend.

17. The process of claim 1, wherein the process further includes a particle size reduction step applied after homogenization, comprising passing the composition through a microfluidization chamber at pressures between 800 and 1,800 bar for 5 to 15 passes, wherein each pass involves acceleration through microchannels having dimensions between 50 and 150 micrometers to achieve a particle size distribution with a polydispersity index below 0.3.

18. The process of claim 1, wherein the process further includes a controlled solvent removal step after blending and homogenization, comprising subjecting the composition to rotary evaporation at a temperature between 35 °C and 45 °C and pressure between 100 and 200 mbar for 20 to 60 minutes, followed by vacuum drying at 40-50 °C for an additional 2 to 6 hours to remove residual organic solvents to levels below 0.5% w/w.

19. The process of claim 1, wherein the process includes incorporation of a stabilizing polysaccharide selected from xanthan gum or hydroxypropyl methylcellulose in a concentration between 0.2% and 1.5% w/w, wherein the polysaccharide is hydrated in purified water for 30 to 90 minutes prior to addition, and subsequently introduced into the composition under stirring at 300 to 700 rpm to increase viscosity and maintain dispersion uniformity, and wherein the combining, homogenization, and stabilization steps are controlled such that the resulting phytotherapeutic composition comprises total boswellic acids in a concentration range of 25% to 65% by weight of the homogeneous bioactive blend, including 3-acetyl-11-keto-β-boswellic acid present in a concentration between 8% and 30% by weight of the homogeneous bioactive blend, and total guggulsterones present in a concentration between 2% and 12% by weight of the homogeneous bioactive blend, wherein the weight ratio of total boswellic acids to total guggulsterones is maintained between 2:1 and 8:1, and wherein the composition further comprises residual solvent content below 0.5% w/w, particle size distribution characterized by a mean diameter below 250 nm with a polydispersity index below 0.35, and a pH maintained between 5.5 and 6.8 through controlled buffering during the stabilization step.
